(19) United States
(12) Patent Application Publication
(10) Pub. No.: US 2025/0237656 A1
(43) Pub. Date: Jul. 24, 2025

(54) METHOD FOR DETECTING AN ANALYTE OF INTEREST IN A SAMPLE

(71) Applicant: Roche Diagnostics Operations, Inc., Indianapolis, IN (US)

(72) Inventors: Bikram Keshari Agrawalla, Bichl (DE); Francesco Ansideri, Muenchen (DE); Dieter Heindl, Muenchen (DE); Lars Hillringhaus, Koenigsdorf (DE); Hannes Kuchelmeister, Muenchen (DE); Giuseppe Prencipe, Koenigsdorf (DE); Sona Simonyiova, Muenchen (DE)

(21) Appl. No.: 18/839,704

(22) PCT Filed: Feb. 16, 2023
(86) PCT No.: PCT/EP2023/053868

§ 371 Date: Aug. 19, 2024

(30) Foreign Application Priority Data
Feb. 18, 2022 (EP) 22157541.8

(51) Int. Cl. G01N 33/58; G01N 33/533; G01N 33/534; G01N 33/535; G01N 33/60

(52) U.S. Cl. CPC G01N 33/581; G01N 33/533; G01N 33/534; G01N 33/535; G01N 33/582; G01N 33/60

(57) ABSTRACT

The present invention relates to a method for determining at least one analyte of interest and the use thereof. The present invention further relates to a kit, a complex, a method to synthesize a complex, a monomer and the use thereof for detecting the analyte of interest in the sample.

### FIELD OF THE INVENTION

The present invention relates to a method for determining at least one analyte of interest and the use thereof. The present invention further relates to a kit, a complex, a method to synthesize a complex, a monomer and the use thereof for detecting the analyte of interest in the sample.

### BACKGROUND OF THE INVENTION

U.S. Pat. No. 9,511,150 reports sugar alcohols and crosslinking reagents, macromolecules, and therapeutic bioconjugates. US 2016/0250896 report phosphonates and sulfonates and hydrophilic linkers, and uses of such linkers for conjugation of drugs to cell binding molecules. US 2010/0009902 reports conjugation with PEG (polyethylene glycol) having a selected molecular weight. Vlahov I. R. et al J. Org. Chem. 75 (2010) 3685-3691 report a carbohydrate-based synthetic approach to control toxicity profiles of folate-drug conjugates. In more detail, the document discloses incorporation of 1-amino-1-deoxy-d-glucitol-γ-glutamate subunits into a peptidic backbone. Synthesis of Fmoc-3,4;5,6-di-O-isopropylidene-1-amino-1-deoxy-d-glucitol-γ-glutamate, suitable for Fmoc-strategy solid-phase peptide synthesis (SPPS), was achieved in four steps from 8-gluconolactone. Addition of alternating glutamic acid and 3,4;5,6-di-O-isopropylidene-1-amino-1-deoxy-d-glucitol-γ-glutamate moieties onto a cysteine-loaded resin, followed by the addition of folate, deprotection, and cleavage, resulted in the isolation of the new folate-spacer: Pte-γGlu-(Glu (1-amino-1-deoxy-d-glucitol)-Glu) 2-Glu (1-amino-1-deoxy-d-glucitol)-Cys-OH.

A particular technical feature known to the art of polymer chemistry is polydispersity which denotes the lack of uniformity in the amount of incorporated monomers and/or polymer chain length. A particular technical problem is posed by frequently observed polydispersity of linker-comprised compounds and conjugates.

Specifically, PEG-based linkers may have such drawbacks. Owing to the technical features of typically used polymerization chemistry, resulting high-molecular-weight PEG molecules are characterized by substantial polydispersity. I.e. a typical polymerization yields a mixture of molecules with different molecular masses. Using such mixed-molecular-weight PEG molecules as linkers leads to a propagation of the polydispersity among the resulting conjugates. As a result, any analysis of the conjugates is complicated as a desired conjugate would be defined a uniform molecular weight. Such uniform molecular weight is however not attained. In addition, despite the hydrophilicity of a PEG moiety in a spacer, certain conjugates with PEG still lack sufficient solubility.

Polysaccharides also tend to be polydisperse and variable in structure, due to the complexity and difficulties of sugar chemistry synthesis of longer and more complex alcohols requires elaborate and low yielding protecting group manipulations.

There is thus an urgent need in the art to overcome the above mentioned problems.

For the present invention, specific substantially monodisperse linker molecules with polyols have been devised which can be used to advantageously crosslink functional molecules. The inventors have found that certain linker molecules with polyols not only offer superior hydrophilicity over PEG-containing derivatives. In an exemplary setting a complex comprising such a linker which crosslinks an analyte-specific binding agent and a label compound produces an improved signal-to-noise ratio in an analyte detection assay. Further, the linker and/or complex shows monodispersity, which preferably results from the peptide synthesis and can be shown by the HPLC chromatograms. The complex and/or monomer described herein is stable.

It is an object of the present invention to provide a method for determining at least one analyte of interest and the use thereof. Further, it is an object of the present invention to provide a kit, a complex, a method to synthesize a complex, a monomer and the use thereof for detecting the analyte of interest in the sample.

This object is or these objects are solved by the subject matter of the independent claims. Further embodiments are subjected to the dependent claims.

### SUMMARY OF THE INVENTION

In the following, the present invention relates to the following apects:

In a first aspect, the present invention relates to a method for detecting an analyte of interest in a sample comprising the steps of
a) Providing the sample comprising the analyte of interest,
b) Providing a complex comprising a linker, wherein the linker covalently binds a label compound and an analyte-specific binding agent, wherein the label compound is capable of producing a detectable signal, preferably a chemiluminescence based signal, more preferably an electrochemiluminescence based signal,
c) Coupling the sample of step a) with the complex of step b),
d) Detecting the analyte of interest by using the detectable signal of the label compound,
wherein the complex is a compound of formula I:

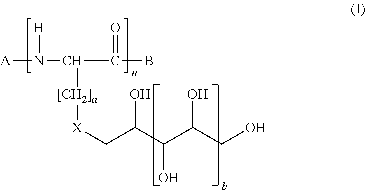

wherein A represents the label compound and B represents the analyte-specific binding agent or vice versa,
X is selected from the group consisting of O, S, CH₂, SO, SO₂, 1,2,3-triazole, and amide,
a is 1 to 5, preferably 1 to 4, more preferably 1 to 2,
b is 0 to 3, and
n is an integer from 1 to 20, preferably 1 to 15, or from 2 to 20, preferably 2 to 15.

In a second aspect, the present invention relates to the use of the method according to the first aspect of the present invention for detecting the analyte of interest in the sample.

In a third aspect, the present invention relates to a kit for performing detection of an analyte of interest in a sample, the kit comprising in separate containers
a) a solid phase capable of immobilizing the analyte;
b) a compound of formula I:

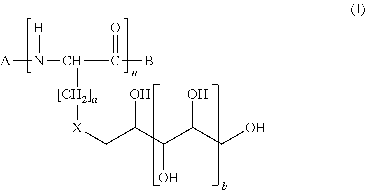

wherein A represents the label compound and B represents the analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20 or from 2 to 20, preferably 1 to 15, more preferably 2 to 15.

In a fourth aspect, the present invention relates to the use of the kit according to the third aspect of the present invention for detecting the analyte of interest in the sample.

In a fifth aspect, the present invention relates a complex of formula I,

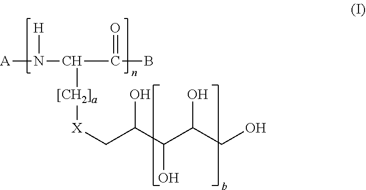

wherein A represents a label compound and B represents an analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15,
preferably wherein the compound is suitable to detect an analyte of interest in a sample.

In a sixth aspect, the present invention relates to a method to a method to synthesize a complex of the fifth aspect of the present invention comprising the steps of
a) Providing a monomer or derivatives thereof, wherein the monomer is an amino acid comprising an amino group, a carboxy group and at least two hydroxyl groups, wherein the amino group or the carboxy group is protected by a first protecting group, and the at least one or each hydroxyl group is protected by a second protecting group,
b) Using the monomer in a process of solid phase peptide synthesis, cleaving the first and second protection group and forming a complex of formula I

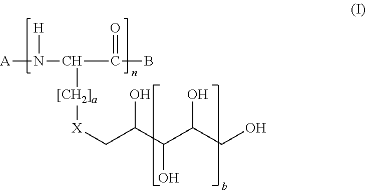

wherein A represents the label compound and B represents the analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15.

In an eight aspect, the present invention relates to a monomer used for a peptide-based synthesis comprising the following formula II

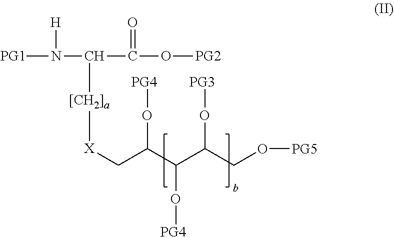

wherein PG1, PG2, PG3, PG4, PG5, PG6 are each independently a protecting group, preferably if PG1 is a protecting group, PG2=H or vice versa,
wherein X is selected from the group consisting of O, S, CH₂, SO, SO₂ or 1,2,3-triazole, and amide,
wherein a is 1 to 5, preferably 1 to 4, more preferably 1 to 2,
wherein b is 0 to 3.

### LIST OF FIGURES

FIG. 1 shows the Elecsys ECL technology.

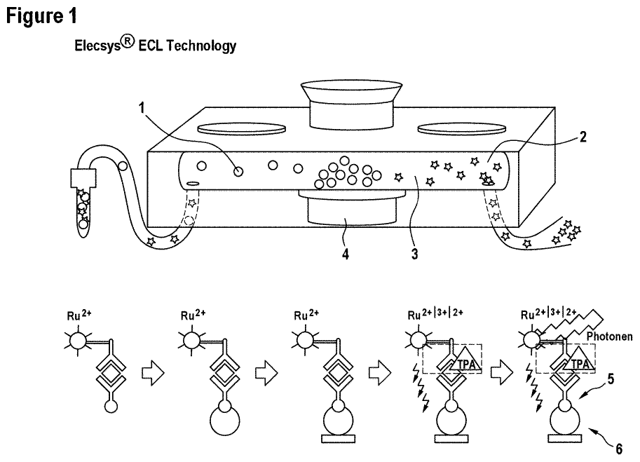

FIG. 2 shows the conjugate stability of different compounds as disclosed herein.

### DETAILED DESCRIPTION OF THE INVENTION

Before the present invention is described in detail below, it is to be understood that this invention is not limited to the particular embodiments and examples described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “detecting” the analyte of interest, as used herein refers to the quantification or qualification of the analyte of interest, e.g. the presence or amount of the analyte of interest in the sample, employing appropriate methods of detection described elsewhere herein.

The term “Coupling” the sample of step a) with the complex of step b) as used herein is a broad term and is to be given its ordinary and customary meaning to a person of ordinary skill in the art and is not to be limited to a special or customized meaning. The term specifically may refer, without limitation, to combining the sample comprising the analyte of interest and the complex. Preferably, coupling or combining means to covalently or non-covalently bind the sample, preferably the analyte, and the complex.

In the context of the present disclosure, the term “analyte”, “analyte molecule”, or “analyte(s) of interest” are used interchangeably referring the chemical species to be analysed via a detectable label. Chemical species suitable to be analysed via a detectable label, i.e. analytes, can be any kind of molecule present in a living organism, include but are not limited to nucleic acid (e.g. DNA, mRNA, miRNA, rRNA etc.), amino acids, peptides, proteins (e.g. cell surface receptor, cytosolic protein etc.), drug molecules, metabolite or hormones (e.g. testosterone, estrogen, estradiol, etc.), fatty acids, lipids, carbohydrates, steroids, ketosteroids, secosteroids (e.g. Vitamin D), molecules characteristic of a certain modification of another molecule (e.g. sugar moieties or phosphoryl residues on proteins, methyl-residues on genomic DNA) or a substance that has been internalized by the organism (e.g. therapeutic drugs, drugs of abuse, toxin, etc.) or a metabolite of such a substance. Such analyte may serve as a biomarker. In the context of present invention, the term “biomarker” refers to a substance within a biological system that is used as an indicator of a biological state of said system. An “analyte” can be any molecule which can be bound by an analyte-specific receptor. In an embodiment, an analyte is an antigen of an infectious agent. Examples of infectious agents are viruses, bacteria and protozoic pathogens that infect humans. In an embodiment, an analyte is a viral antigen, in an embodiment a hepatitis virus antigen or a human retroviral antigen. In an embodiment, an analyte is a hepatitis C virus or hepatitis B virus or HIV antigen.

Generally, the term “receptor” denotes any compound or composition capable of recognizing a particular spatial and polar organization of a target molecule i.e. an epitopic site of an analyte. Thus, the term “analyte-specific receptor” as referred to herein includes analyte-specific reactants capable of binding to or complexing an analyte. This includes but is not limited to antibodies, specifically monoclonal antibodies or antibody fragments. Such a receptor can act as a catcher of the analyte, e.g. to immobilize the analyte. An epitope recognized by the antibody is bound, followed by labeled antibodies specific to another epitope of the analyte. Other receptors are known to those of skill in the art. The particular use of various receptors in a receptor-based analyte assay will be understood by those of skill in the art with reference to this disclosure.

Analytes or an analyte of interest may be present in a sample, e.g. in a biological or clinical sample. The term “biological or clinical sample” are used interchangeably herein, referring to a part or piece of a tissue, organ or individual, typically being smaller than such tissue, organ or individual, intended to represent the whole of the tissue, organ or individual. Upon analysis a biological or clinical sample provides information about the tissue status or the health or diseased status of an organ or individual. Examples of biological or clinical samples include but are not limited to fluid samples such as blood, serum, plasma, synovial fluid, spinal fluid, urine, saliva, and lymphatic fluid, or solid biological or clinical samples such as dried blood spots and tissue extracts. Further examples of biological or clinical samples are cell cultures or tissue cultures.

In context of the present disclosure, the term “antibody” relates to full immunoglobulin molecules, specifically IgMs, IgDs, IgEs, IgAs or IgGs, as well as to parts of such immunoglobulin molecules, like Fab-fragments or V_{L}-, V_{H}- or CDR-regions. Furthermore, the term relates to modified and/or altered antibody, like chimeric and humanized antibodies. The term also relates to modified or altered monoclonal or polyclonal antibodies as well as to recombinantly or synthetically generated/synthesized antibodies. The term also relates to intact antibodies as well as to antibody fragments/parts thereof, like, separated light and heavy chains, Fab, Fab/c, Fv, Fab′, F(ab′)₂. The term “antibody” also comprises antibody derivatives, bifunctional antibodies and antibody constructs, like single chain Fvs (scFv), bispecific scFvs or antibody-fusion proteins.

In chemistry, “solid-phase synthesis” is a method in which molecules are covalently bound on a solid support material and synthesised step-by-step in a single reaction vessel utilising selective protecting group chemistry. As a specific embodiment, solid phase peptide synthesis is a common technique involving discrete steps for the synthesis of peptides. This approach permits unreacted reagents to be removed by washing without loss of product. Usually, peptides are synthesised from the carbonyl group side (C-terminus) to amino group side (N-terminus) of the amino acid chain. In peptide synthesis, an amino-protected amino acid is bound to a solid phase material such as, but not limited to, polystyrene beads, thereby forming a covalent bond between the carbonyl group and the resin, most often an amido or an ester bond. Then the amino group is deprotected and reacted with the carbonyl group of the next amino-protected amino acid. The solid phase now bears a dipeptide. This cycle is repeated to form the desired peptide chain. After all reactions are complete, the synthesised peptide is cleaved from the solid phase.

More specifically, the carboxyl moiety of each incoming amino acid is activated by one of several strategies and couples with the α-amino group of the preceding amino acid. The α-amino group of the incoming residue is temporarily blocked in order to prohibit peptide bond formation at this site. The residue is de-blocked at the beginning of the next synthesis cycle. In addition, reactive side chains on the amino acids are modified with appropriate protecting groups. The peptide chain is extended by reiteration of the synthesis cycle. Excess reagents are used to drive reactions as close to completion as possible.

The “blocking group” or “protecting group” or “protection group” used for blocking the α-amino group determines both the synthesis chemistry employed and the nature of the side-chain protecting groups. The two most commonly used α-amino protecting groups are Fmoc (9-fluorenyl-methoxy-carbonyl) and Boc (tert-butoxycarbonyl). The protection of reactive groups in the side chains is provided by protecting groups which are orthogonal to the protecting group used for the α-amino group, which include but are not limited to carbamate, ether, ester, amide, acetal, enamine.

After fully assembling the peptide the side-chain protecting groups are removed, if so desired, and the peptide is cleaved from the solid support, using conditions that inflict minimal damage on labile residues.

The product can be analyzed to verify the sequence thereafter. A synthetic peptide is usually purified by gel chromatography or HPLC.

Coupling of a label and/or a target molecule to the peptide are possible by different methods. As a non-limiting example, a building block amenable to SPPS may be incorporated into the peptide, wherein the building block comprises a reactive group which is optionally protected and which can be used for forming a linkage with a further compound of choice after the SPPS process. Alternatively, the compound of choice may already be attached to the building block when it enters the SPPS process. Other alternatives are possible.

The Fmoc protecting group is base-labile. It is usually removed with a dilute base such as piperidine. The side-chain protecting groups are removed by treatment with trifluoroacetic acid (TFA), which also cleaves the bond anchoring the peptide to the support. The Boc protecting group is removed with a mild acid (usually dilute TFA). Hydrofluoric acid (HF) can be used both to deprotect the amino acid side chains and to cleave the peptide from the resin support. Fmoc is a gentler method than Boc since the peptide chain is not subjected to acid at each cycle and has become the major method employed in commercial automated peptide synthesis.

The protecting groups for the amino groups mostly used in the peptide synthesis are 9-fluorenylmethyloxycarbonyl group (Fmoc) and t-butoxycarbonyl (Boc). A number of amino acids bear functional groups in the side chain which must be protected specifically from reacting with the incoming N-protected amino acids. In contrast to Boc and Fmoc groups, these have to be stable over the course of peptide synthesis although they are also removed during the final deprotection of peptides.

A “label compound” includes a moiety that is detectable or that can be rendered detectable. The skilled person knows a label as a compound or composition capable of providing a detectable signal in conjunction with physical activation (or excitation) or chemical reagents and capable of being modified, so that the particular signal is diminished or increased.

Specific embodiments of a label compound, which is capable of producing a detectable signal, include labels which are detectable by a number of commercially available instruments that utilize chemiluminescence, preferably electrochemiluminescence (ECL) for analytical measurements. Species that can be induced to emit ECL (ECL-active species) have been used as ECL labels. Examples of ECL labels include: i) organometallic compounds where the metal is from, for example, the noble metals of group VIII, including Ru-containing, Ir-containing and/or Os-containing organometallic compounds such as the tris-bipyridyl-ruthenium (BPRu) moiety and ii) luminol and related compounds. Species that participate with the ECL label in the ECL process are referred to herein as ECL coreactants. Commonly used coreactants include tertiary amines (e.g., see U.S. Pat. No. 5,846,485), oxalate, and persulfate for ECL from RuBpy and hydrogen peroxide for ECL from luminol (see, e.g., U.S. Pat. No. 5,240,863). The light generated by ECL labels can be used as a reporter signal in diagnostic procedures (Bard et al., U.S. Pat. No. 5,238,808). For instance, an ECL label can be covalently coupled to a binding agent such as an antibody, nucleic acid probe, receptor or ligand; the participation of the binding reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL coreactants). For more background on ECL, ECL labels, ECL assays and instrumentation for conducting ECL assays see U.S. Pat. Nos. 5,093,268; 5,147,806; 5,324,457; 5,591,581; 5,597,910; 5,641,623; 5,643,713; 5,679,519; 5,705,402; 5,846,485; 5,866,434; US5, 786, 141; U.S. Pat. Nos. 5,731,147; 6,066,448; U.S. Pat. Nos. 6,136,268; 5,776,672; 5,308,754; 5,240,863; 6,207,369 and 5,589,136; and WO99/63347, WO00/03233, WO99/58962, WO99/32662, WO99/14599, WO98/12539, WO97/36931 and WO98/57154.

The term “chemiluminescence based signal” refers to a signal that is produced by the emission of light (luminescence), as the result of a chemical reaction. This signal is detectable, e.g. by a number of commercially available instruments that utilize chemiluminescence. The term “electrochemiluminescence based signal” refers to a signal that consists of the emission of light (e.g. luminescence), as the result of an electrochemical reaction, wherein the excited state of one species can be obtained at an electrode.

In the context of the present disclosure, the term “complex” refers to the product produced by the reaction of a linker, a label compound and an analyte-specific binding agent. This reaction can lead to the formation of a covalent bond between the label compound and the linker on one side and the linker and the analyte-specific binding agent on the other side.

The term “linker” can refer to a compound serving as a spacer between the label compound and the analyte-specific binding agent and/or influencing the physico-chemical properties of the complex such as hydrophilicity and solubility.

The term “analyte-specific binding agent” refers to a (macro) molecule (protein, peptide, nucleic acid, etc.) capable of specifically binding the analyte of interest, e.g. a monoclonal antibody.

The term “A represents the label compound and B represents the analyte-specific binding agent or vice versa” means that A of formula I represents the label compound and B of formula I represents the analyte-specific binding agent. Alternatively, it means that that B of formula I represents the label compound and A of formula I represents the analyte-specific binding agent.

The term “Coupling the sample of step a) with the complex of step b)” refers to the reaction of the sample comprising or containing the analyte of interest with the complex of step b). Preferably, coupling refers to a non-covalent binding between the sample, preferably the analyte of interest, and the complex.

The term “peptide” means a molecule that is formed using naturally occurring L-amino acids or analogs thereof, like D-amino acids or N-alkylated amino acids or the like. Preferred amino acids are selected from the group consisting of Ala, Arg, Asn, Asp, Cys, Glu, Gln, Gly, His, Hyl, Hyp, Ile, Leu, Lys, Met, Phe, Pro, Ser, Thr, Trp, Tyr, and Val. Also other building blocks are possible having a carboxylic acid and an amino group. Additionally, modifications like fluorescence dyes or biotin are possible.

“Solid phase peptide synthesis (SPPS)” is a well established method. Merrifield et al. were the first who developed a convenient strategy for the build up of peptides by subsequently coupling amino acid monomers using a solid phase resin as a heterogeneous reaction medium (R. B. Merrifield, J. Am. Chem. Soc. 85 (1963) 2149-2154).

As a major advantage in comparison with the in-solution synthesis of peptides SPPS can be automated easily and impurities or by-products, reagents as well as unreacted starting material can be washed away while the product or intermediate remains tethered on the solid phase.

Normally the abovementioned Merrifield method starts with the attachment of the first C-terminal amino acid to a so called “linker” of a crosslinked polystyrene resin. The “linker” serves as a bridging element between the resin and the C-terminal amino acid of the peptide to be synthesized and the linker contains an acid sensitive bond to be used for the detachment of the peptide after synthesis.

As an example for a typical SPPS protocol the N-terminus can be protected with the 9-fluorenylmethoxycarbonyl (Fmoc) group, which is stable in acid, but removable by base. Any side chain functional groups are protected with base stable groups to make sure that only the N-terminal amino group incorporated in the peptide backbone can react-after removal of the Fmoc group—with the carboxylic acid group of the subsequent amino acid. As already mentioned the first step after the immobilization of the first amino acid is the deprotection of the amino function by removal of the Fmoc group using 20% piperidine in N,N-dimethylformamide (DMF). The amino function is coupled with an activated carboxylic acid via O-(Benzotriazol-1-yl)-N,N,N′,N′-tetramethyluronium hexafluorphosphate (HBTU) ester of the next amino acid in the presence of a base to form a new amide bond. This process is repeated until the desired peptide is assembled at the resin. As a last step the complete peptide is cleaved from the resin using a solution containing trifluoroacetic acid (TFA). The released peptide in the solution can be precipitated and washed before further purification.

This “classic” method for SPPS was optimized in recent years using modified resins, linkers, protective groups, coupling chemistries and cleavage procedures but the principle remains the same.

The term “solid phase” as used herein refers to a wide variety of materials including solids, semi-solids, gels, films, membranes, meshes, felts, composites, particles, resins, papers and the like typically used by those of skill in the art to sequester molecules. The solid phase can be a material, e.g. in a chromatographic column or as a specific embodiment a functionalized resin in a column of a device for solid phase synthesis. The solid phase can be non-porous or porous. The solid phase can be non-magnetic or magnetic (encompassing diamagnetic, paramagnetic, and superparamagnetic features).

Surfaces of solid phases as those described above may be modified to provide linkage sites, for example by bromoacetylation, silation, addition of amino groups using nitric acid, and attachment of intermediary proteins, dendrimers and/or star polymers. This list is not meant to be limiting, and any method known to those of skill in the art may be employed.

The term “polyol-units” refers to monomers (e.g. amino acids) comprising 1 or more OH groups. Such monomers can be covalently linked to each other to form a homo- or heteropolymer.

A “kit” is any manufacture (e.g., a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like. In particular, each of the container means comprises one of the separate elements to be used in the method of the first aspect. Kits may further comprise one or more other reagents including but not limited to reaction catalyst. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

In this detailed description, references to “one embodiment”, “an embodiment”, or “in embodiments” mean that the feature being referred to is included in at least one embodiment of the technology with regards to all its aspects according to present disclosure. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the technology in all its aspects according to present disclosure can include any variety of combinations and/or integrations of the embodiments described herein.

### EMBODIMENTS

In a first aspect, the present invention relates to method for detecting an analyte of interest in a sample comprising the steps of:
a) Providing the sample comprising the analyte of interest,
b) Providing a complex comprising a linker, wherein the linker covalently binds a label compound and an analyte-specific binding agent, wherein the label compound is capable of producing a detectable signal, preferably a chemiluminescence based signal, more preferably an electrochemiluminescence based signal,
c) Coupling the sample of step a) with the complex of step b),
d) Detecting the analyte of interest by using the detectable signal of the label compound,
wherein the complex is a compound of formula I:

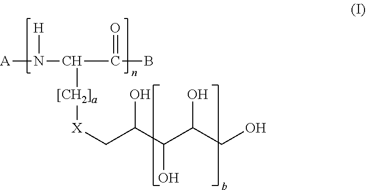

wherein A represents the label compound and B represents the analyte-specific binding agent or vice versa,
X is selected from the group consisting of O, S, CH₂, SO, SO₂, 1,2,3-triazole, and amide,
a is 1 to 5, preferably 1 to 4, more preferably 1 to 2,
b is 0 to 3, and
n is an integer from 1 to 20, preferably 1 to 15, or from 2 to 20, preferably 2 to 15.

The inventors surprisingly found that subject matters of the present invention, in particular the method according to the first aspect of the invention, show a complex comprising in particular a peptide-based polyol linkers, with extremely good control on structure and polydispersity. Using a single molecular weight and pure linker decreases complexity of product purification, characterization and improves reproducibility of manufacturing. In particular, the solid-phase peptide chemistry can be utilized to give a complex of the present invention. Further, the complex is stable.

The method as referred to in accordance with the present invention includes a method which essentially consists of the aforementioned steps or a method which includes further steps. Moreover, the method of the present invention, preferably, is an ex vivo and more preferably an in vitro method. Moreover, it may comprise steps in addition to those explicitly mentioned above. For example, further steps may relate to the detecting of further analytes of interests and/or to sample pre-treatments, enrichment steps or evaluation of the results obtained by the method. The method may be carried out manually or assisted by automation. Preferably, step (a), (b), (c) and/or (d) may in total or in part be assisted by automation, e.g., by a suitable robotic and sensory equipment.

According to step (a), the sample is provided.

According to step b), a complex is provided. The complex is a compound of formula I. The complex comprises a linker. The linker covalently binds a label compound and an analyte-specific binding agent. The label compound is capable of producing a detectable signal. Preferably, the detectable label is a chemiluminescence based signal, more preferably an electrochemiluminescence based signal.

According to step c), the sample of step a) with the complex of step b) is coupled.

According to step d), the analyte of interest is detected by using the detectable signal of the label compound.

In embodiments of the first aspect of the invention, X=O, a=1 and b=0.

In embodiments of the first aspect of the invention, X=O, a=1 and b=1.

In embodiments of the first aspect of the invention, X=O, a=1 and b=2.

In embodiments of the first aspect of the invention, X=O, a=1 and b=3.

In embodiments of the first aspect of the invention, X=O, a=2 and b=0.

In embodiments of the first aspect of the invention, X=O, a=2 and b=1.

In embodiments of the first aspect of the invention, X=O, a=2 and b=2.

In embodiments of the first aspect of the invention, X=O, a=2 and b=3.

In embodiments of the first aspect of the invention, X=O, a=3 and b=0.

In embodiments of the first aspect of the invention, X=O, a=3 and b=1.

In embodiments of the first aspect of the invention, X=O, a=3 and b=2.

In embodiments of the first aspect of the invention, X=O, a=3 and b=3.

In embodiments of the first aspect of the invention, X=O, a=4 and b=0.

In embodiments of the first aspect of the invention, X=O, a=4 and b=1.

In embodiments of the first aspect of the invention, X=O, a=4 and b=2.

In embodiments of the first aspect of the invention, X=O, a=4 and b=3.

In embodiments of the first aspect of the invention, X=1,2,3-triazole, a=1 and b=0.

In embodiments of the first aspect of the invention, X=1,2,3-triazole, a=1 and b=1.

In embodiments of the first aspect of the invention, X=1,2,3-triazole, a=1 and b=2.

In embodiments of the first aspect of the invention, X=1,2,3-triazole, a=1 and b=3.

In embodiments of the first aspect of the invention, X=1,2,3-triazole, a=2 and b=0.

In embodiments of the first aspect of the invention, X=1,2,3-triazole, a=2 and b=1.

In embodiments of the first aspect of the invention, X=1,2,3-triazole, a=2 and b=2.

In embodiments of the first aspect of the invention, X=1,2,3-triazole, a=2 and b=3.

In embodiments of the first aspect of the invention, X=1,2,3-triazole, a=3 and b=0.

In embodiments of the first aspect of the invention, X=1,2,3-triazole, a=3 and b=1.

In embodiments of the first aspect of the invention, X=1,2,3-triazole, a=3 and b=2.

In embodiments of the first aspect of the invention, X=1,2,3-triazole, a=3 and b=3.

In embodiments of the first aspect of the invention, X=1,2,3-triazole, a=4 and b=0.

In embodiments of the first aspect of the invention, X=1,2,3-triazole, a=4 and b=1.

In embodiments of the first aspect of the invention, X=1,2,3-triazole, a=4 and b=2.

In embodiments of the first aspect of the invention, X=1,2,3-triazole, a=4 and b=3.

In embodiments of the first aspect of the invention, X=amide, a=1 and b=0.

In embodiments of the first aspect of the invention, X=amide, a=1 and b=1.

In embodiments of the first aspect of the invention, X=amide, a=1 and b=2.

In embodiments of the first aspect of the invention, X=amide, a=1 and b=3.

In embodiments of the first aspect of the invention, X=amide, a=2 and b=0.

In embodiments of the first aspect of the invention, X=amide, a=2 and b=1.

In embodiments of the first aspect of the invention, X=amide, a=2 and b=2.

In embodiments of the first aspect of the invention, X=amide, a=2 and b=3.

In embodiments of the first aspect of the invention, X=amide, a=3 and b=0.

In embodiments of the first aspect of the invention, X=amide, a=3 and b=1.

In embodiments of the first aspect of the invention, X=amide, a=3 and b=2.

In embodiments of the first aspect of the invention, X=amide, a=3 and b=3.

In embodiments of the first aspect of the invention, X=amide, a=4 and b=0.

In embodiments of the first aspect of the invention, X=amide, a=4 and b=1.

In embodiments of the first aspect of the invention, X=amide, a=4 and b=2.

In embodiments of the first aspect of the invention, X=amide, a=4 and b=3.

In embodiments of the first aspect of the invention, a is 1.

In embodiments of the first aspect of the invention, a is 2.

In embodiments of the first aspect of the invention, a is 3.

In embodiments of the first aspect of the invention, a is 4.

In embodiments of the first aspect of the invention, n is an integer from 1 to 20, preferably 1 to 15.

In embodiments of the first aspect of the invention, n is an integer from 2 to 20, preferably 2 to 15. In embodiments of the first aspect of the invention, n is an integer. n is selected from the range of 1 to 20. Preferably, n is more than or equal to 2, e.g. 2 or 3 or 4 or 5 or 6 or 7 or 8 or 9 or 10 or 11 or 12 or 13 or 14 or 15 or 16 or 17 or 18 or 19 or 20.

In embodiments of the first aspect of the invention, step b) comprises a peptide-based synthesis, preferably a solid phase peptide synthesis (SPPS).

In embodiments of the first aspect of the invention, the label compound is selected from the group consisting of an enzyme, a fluorescent dye, a luminescent dye, a metal chelate complex, and a moiety containing a radioisotope.

In embodiments of the first aspect of the invention, label compound is capable of being induced to luminesce when electrochemically oxidized or reduced.

In embodiments of the first aspect of the invention, the label compound comprises a metal ion, which is Ru²⁺ or Ir³⁺.

Preferably, the label compound is selected from the following group: Ru or Ir.

In embodiments of the first aspect of the invention, the label compound is covalently bonded to the linker via a first conjugation method, wherein the first conjugation method is selected from the following group: click chemistry, amide, ester, imide, carbonate, carbamate, squarate, thiazole, thiazolidine, hydrazone, oxime, dihydropyridazine, thiol-maleimide, cycloaddition, tetrazine ligation, photoclick, Staudinger ligation, Diels-Alder, cross-coupling, Pictet-Spengler, quadricylcane.

In embodiments of the first aspect of the invention, the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein.

In embodiments of the first aspect of the invention, the analyte-specific binding agent is covalently bonded to the linker via a second conjugation method, wherein the second conjugation method is selected from the following group: click chemistry, amide, ester, imide, carbonate, carbamate, squarate, thiazole, thiazolidine, hydrazone, oxime, dihydropyridazine, thiol-maleimide, cycloaddition, tetrazine ligation, photoclick, Staudinger ligation, Diels-Alder, cross-coupling, Pictet-Spengler, quadricyclane.

Preferably, the analyte-specific binding agent is selected from the following group: antibody, Fab.

In embodiments of the first aspect of the invention, A of formula I represents the label compound and B of formula I represents the analyte-specific binding agent.

In embodiments of the first aspect of the invention, B of formula I represents the label compound and A of formula I represents the analyte-specific binding agent.

In embodiments of the first aspect of the invention, prior, during or after step (c) the analyte is immobilized on a solid phase.

In embodiments of the first aspect of the invention, the sample is selected from the group consisting of sputum, saliva, liquor, urine, whole blood, hemolyzed whole blood, serum and plasma.

In embodiments of the first aspect of the invention, the complex of step (b) is provided in dissolved form, and step (c) is performed in a liquid aqueous buffer.

In embodiments of the first aspect of the invention, the liquid aqueous buffer is selected from phosphate, tris buffer, citrate, cacodylate, barbital, glycine, HEPES, MES, PIPES, MOPS, bis-tris methane, ADA, bis-tris propane, ACES, MOPSO, BES, AMPB, TES, DIPSO, MOBS, acetamidoglycine, TAPSO, TEA, POPSO, HEPPSO, EPS, HEPPS, tricine, gycinamide, Gly-Gly, HEPBS, bicine, TAPS and mixtures thereof.

In embodiments of the first aspect of the invention, the liquid aqueous buffer is selected from phosphate, tris(hydroxymethyl)aminomethane (TRIS, preferably with a pH 6.0-7.4) and mixtures thereof.

In embodiments of the first aspect of the invention, the complex is selected from the groups consisting of at least one compound having at least one of the following formulae 4-I to 4-X:

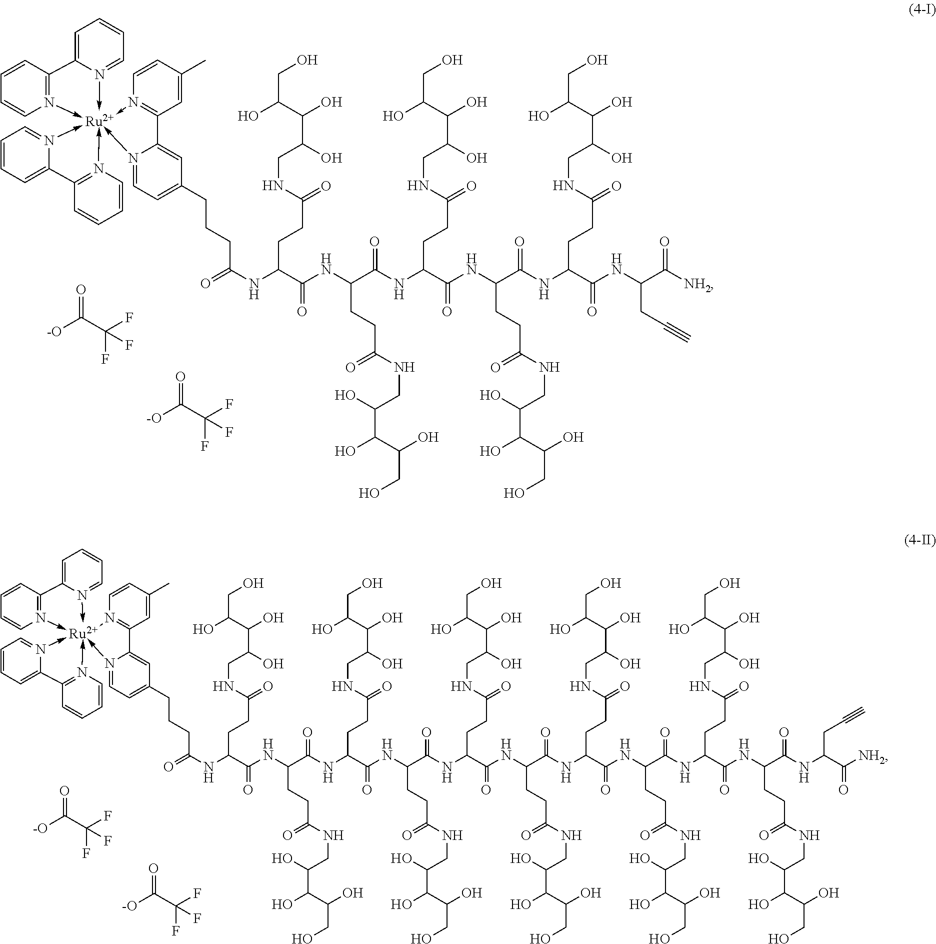

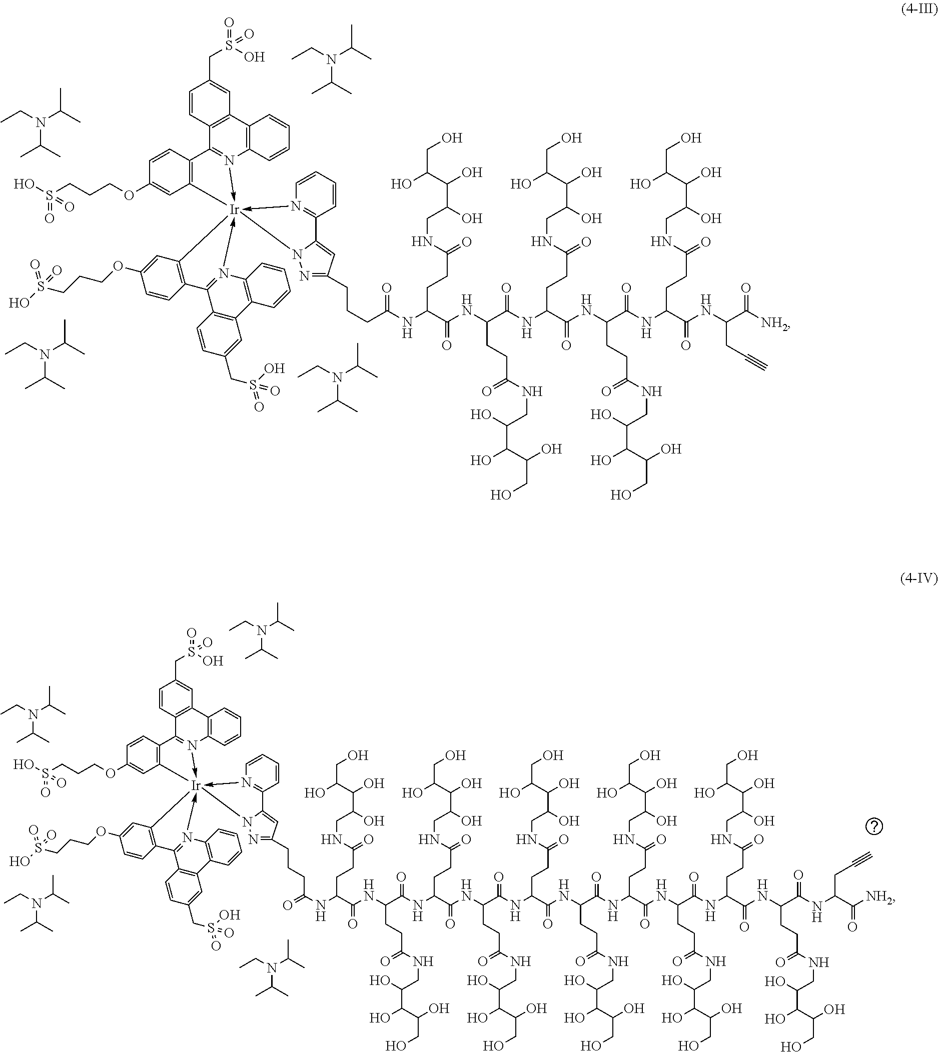

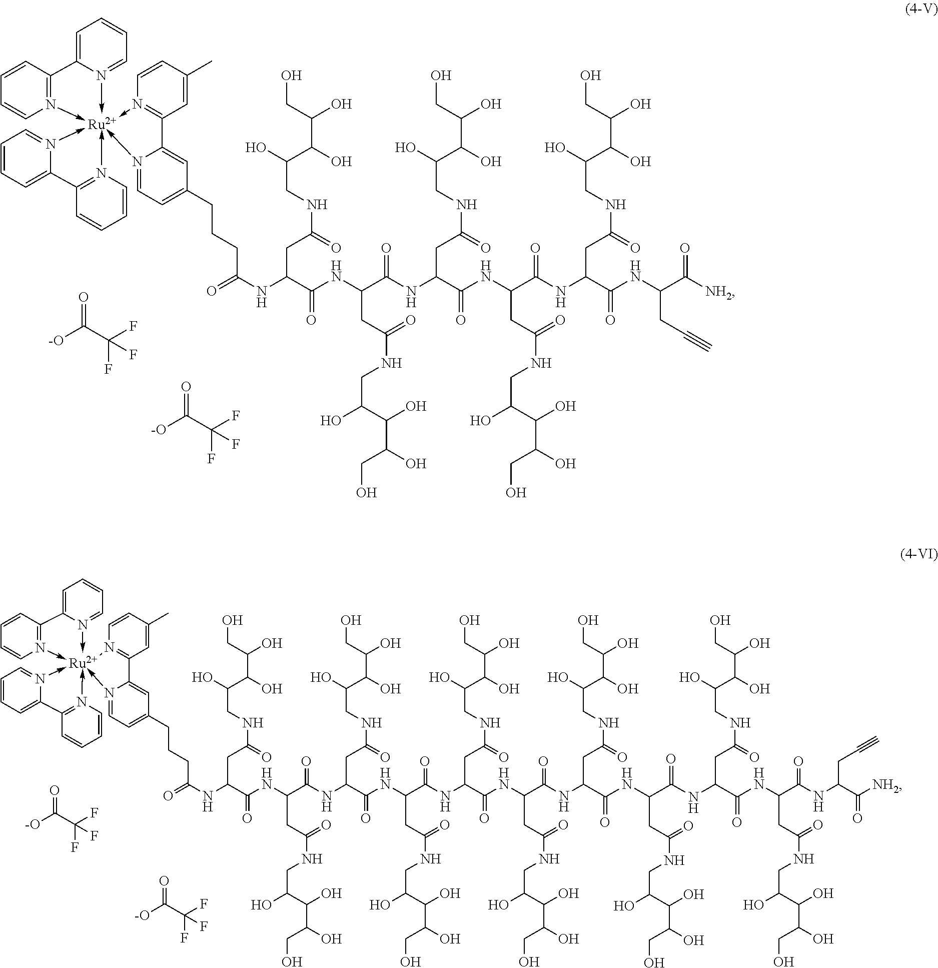

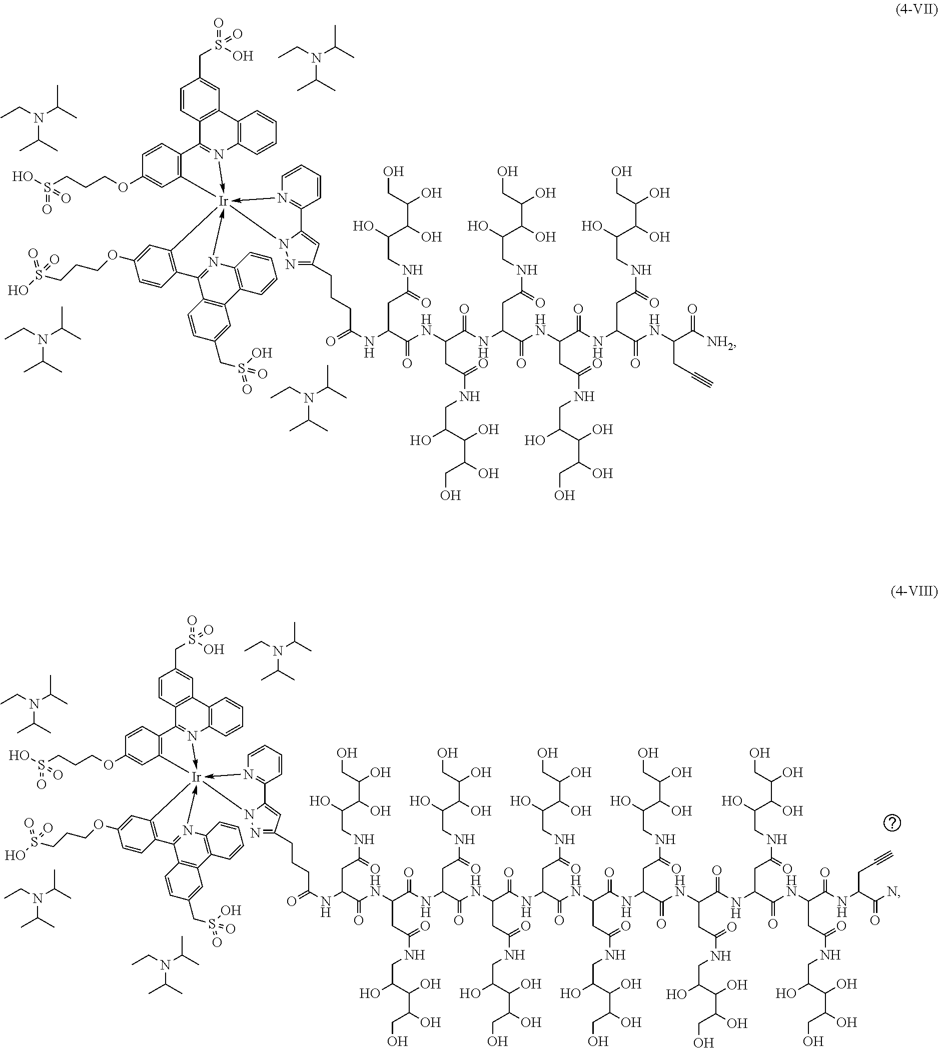

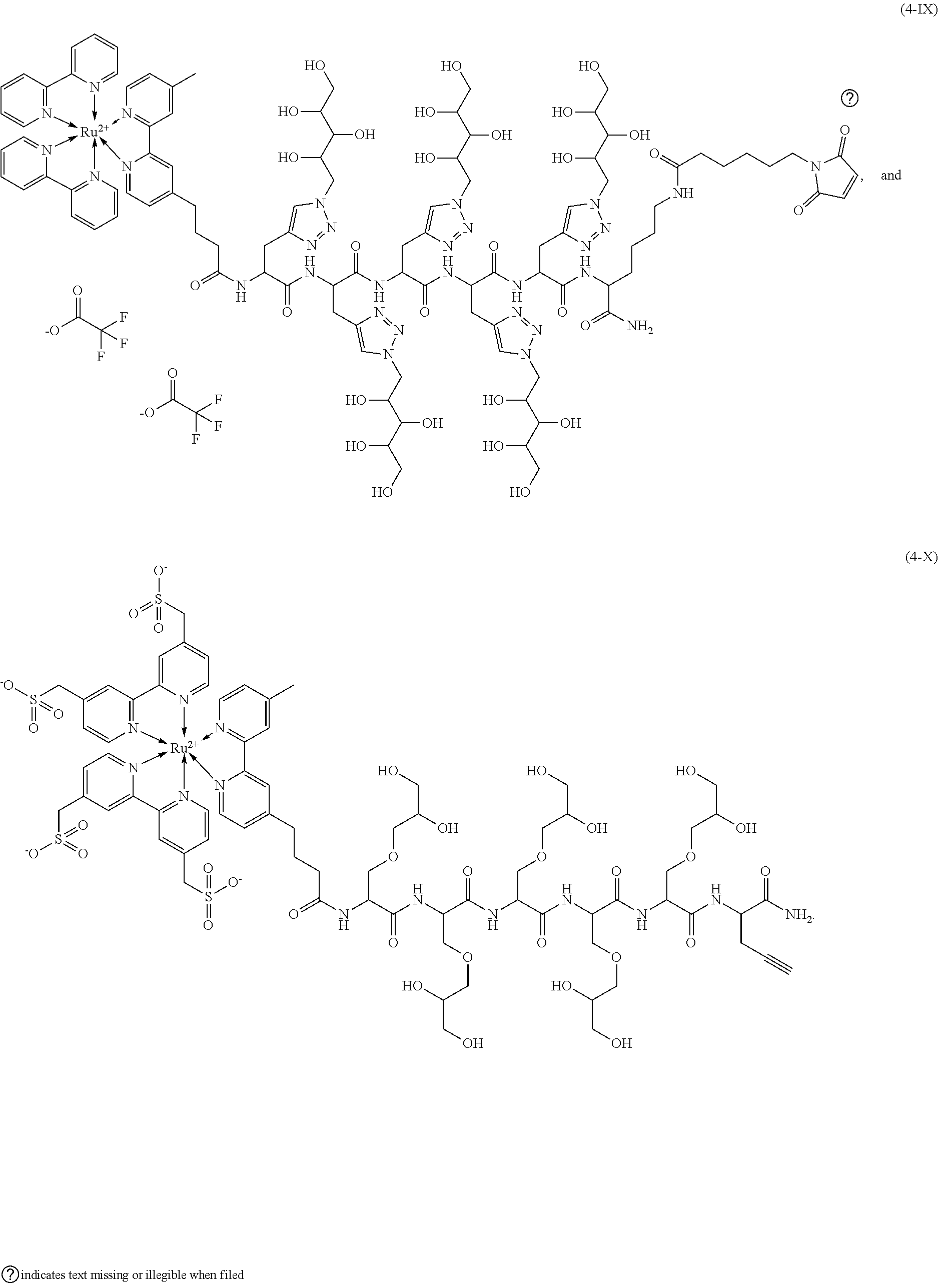

In embodiments, the label compound does not include or is free of a folic acid or derivatives thereof.

In embodiments, the label compound does not include or is free of a folate receptor binding ligand.

In embodiments, the analyte specific binding agent does not include or is free of a cysteine.

In embodiments, the method is free of a drug delivery purpose. This can mean that the method is not aimed at improving drug delivery or the purpose of the method is not related to drug delivery.

In embodiments, the method is a diagnostic method, preferably an in-vitro diagnostic method.

In embodiments, n>2.

In embodiments, n>4.

In embodiments, the label compound is free of a drug compound, e.g. desacetyl vinblastine hydrazide or derivatives thereof.

In a second aspect, the present invention relates to the use of the method according to first aspect of the present invention for detecting the analyte of interest in the sample.

All embodiments mentioned for the first aspect of the invention apply for the second aspect of the invention and vice versa.

In a third aspect, the present invention relates to a kit for performing detection of an analyte of interest in a sample, the kit comprising in separate containers
a) a solid phase capable of immobilizing the analyte;
b) a compound of formula I:

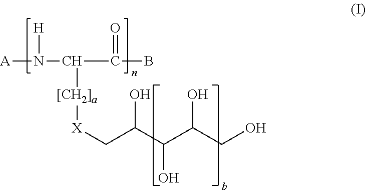

wherein A represents the label compound and B represents the analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15.

All embodiments mentioned for the first aspect of the invention and/or second aspect of the invention apply for the third aspect of the invention and vice versa.

In embodiments of the third aspect of the invention, the complex is embodied in dissolved form.

In embodiments of the third aspect of the invention, the at least one container or containers is made of glass or plastic.

In a fourth aspect, the present invention relates to the use of the kit according to third aspect of the present invention for detecting the analyte of interest in the sample.

All embodiments mentioned for the first aspect of the invention and/or second aspect of the invention and/or third aspect of the invention apply for the fourth aspect of the invention and vice versa.

In a fifth aspect, the present invention relates to a complex of formula I,

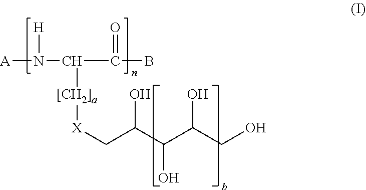

wherein A represents a label compound and B represents an analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15,
preferably wherein the compound is suitable to detect an analyte of interest in a sample.

All embodiments mentioned for the first aspect of the invention and/or second aspect of the invention and/or third aspect of the invention and/or fourth aspect of the invention apply for the fifth aspect of the invention and vice versa.

In embodiments of the fifth aspect of the invention, A or B is selected from the group consisting of a peptide, a polypeptide, and a protein.

In embodiments of the fifth aspect of the invention, A comprises the analyte-specific binding agent and B comprises the label compound, or B comprises the analyte-specific binding agent and A comprises the label compound.

In embodiments of the fifth aspect of the invention, the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein, and the label compound is selected from the group consisting of enzyme, a fluorescent dye, a luminescent dye, a metal chelate complex, and a moiety containing a radioisotope.

In a sixth aspect, the present invention relates to a method to synthesize a complex of the fifth aspect of the invention comprising the steps of
a) Providing a monomer or derivatives thereof, wherein the monomer is an amino acid comprising an amino group, a carboxy group and at least one or at least two hydroxyl groups, wherein the amino group or the carboxy group is protected by a first protecting group, and the at least one or each hydroxyl group is protected by a second protecting group,
b) Using the monomer in a process of solid phase peptide synthesis, cleaving the first and second protection group and forming a complex of formula I

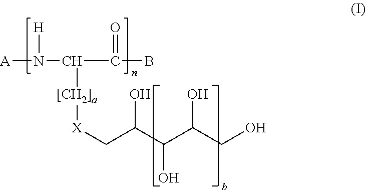

wherein A represents the label compound and B represents the analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15.

All embodiments mentioned for the first aspect of the invention and/or second aspect of the invention and/or third aspect of the invention and/or fourth aspect of the invention and/or fifth aspect of the invention apply for the sixth aspect of the invention and vice versa.

In embodiments of the sixth aspect of the invention, the order of the method steps can vary depending on the metal-complex (e.g. Ru or Ir) which is added. In case of Ru, the peptide is synthesized, the first protective group is cleaved, the Ru label is coupled, the second protective group is cleaved with simultaneous cleavage of the peptide from the solid phase, and finally the analyte-binding agent is coupled. In case of Ir, the peptide is synthesized, the first and second protective groups are cleaved, alongside with the cleavage of the peptide from the solid phase. Finally the Ir label is coupled and then the analyte-binding agent is coupled.

In embodiments of the sixth aspect of the invention, the first and/or second the first and/or second protecting group is selected from the group ester, ether, silylether, acetal, 9-fluorenylmethyloxycarbonyl (Fmoc), tert-butoxycarbonyl (Boc), benzyloxycarbonyl (Cbz), allyloxycarbonyl (Alloc), amide and tert butyl ester.

In embodiments of the sixth aspect of the invention, the first and/or second protecting group or further protecting group is selected from the group consisting of Fmoc, tBu (tert-butyl), Boc (tert-butoxycarbonyl), ether, ester and acetal.

In embodiments of the sixth aspect of the invention, a monomer or a derivative thereof is selected from the following formulae m-1 to m-3:

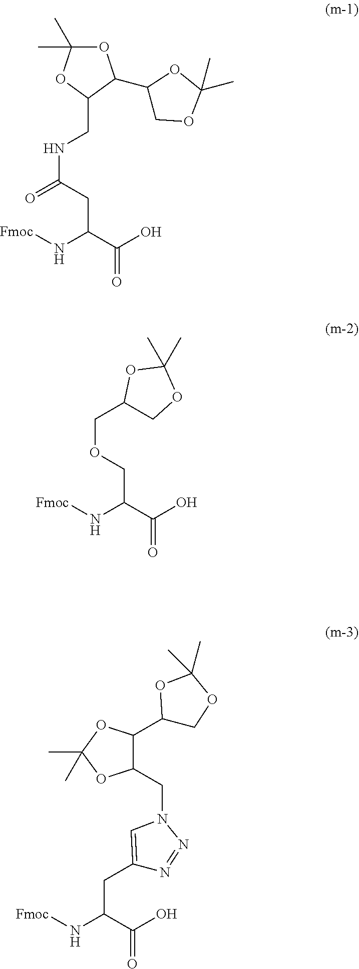

wherein Fmoc means a 9-fluorenylmethoxycarbonyl protecting group.

In a seventh aspect, the present invention relates to monomer used for a peptide-based synthesis comprising the following formula II

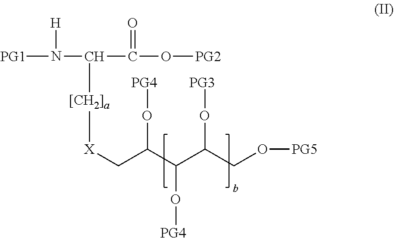

wherein PG1, PG2, PG3, PG4, PG5, PG6 are each independently a protecting group,
preferably if PG1 is a protecting group, PG2=H or vice versa,
wherein X is selected from the group consisting of O, S, CH₂, SO, SO₂ or 1,2,3-triazole, and amide,
wherein a is 1 to 5, preferably 1 to 4, more preferably 1 to 2,
wherein b is 0 to 3.

All embodiments mentioned for the first aspect of the invention and/or second aspect of the invention and/or third aspect of the invention and/or fourth aspect of the invention and/or fifth aspect of the invention and/or sixth aspect of the invention apply for the seventh aspect of the invention and vice versa.

In embodiments of the seventh aspect of the invention, the protecting groups PG1, PG2, PG3, PG4, PG5, PG6 are independently selected from the group consisting of Fmoc, tBu, Boc, ether, ester and acetal.

In embodiments of the seventh aspect of the invention, at least two of the protecting groups selecting from the group consisting of PG1, PG2, PG3, PG4, PG5, PG6 are linked to each other, for example by at least one covalent bond, preferably a single, double or tripe covent bond.

In embodiments of the seventh aspect of the invention, a monomer or a derivative thereof is selected from the following formulae m-1 to m-3:

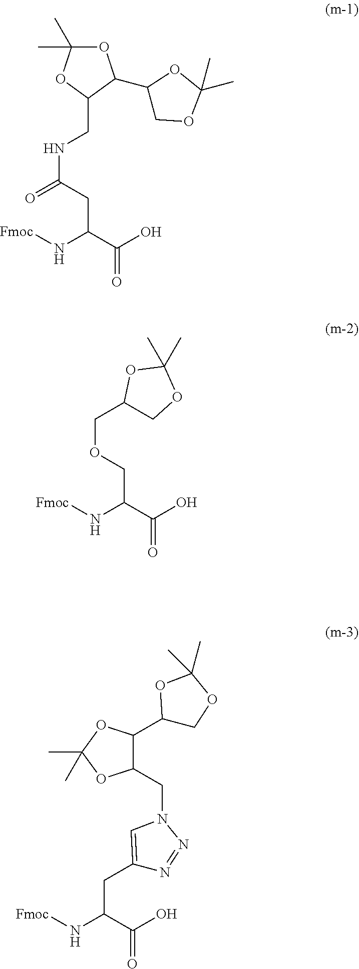

In further embodiments, the present invention relates to the following aspects:

1. A method for detecting an analyte of interest in a sample comprising the steps of
a) Providing the sample comprising the analyte of interest,
b) Providing a complex comprising a linker, wherein the linker covalently binds a label compound and an analyte-specific binding agent, wherein the label compound is capable of producing a detectable signal, preferably a chemiluminescence based signal, more preferably an electrochemiluminescence based signal,
c) Coupling the sample of step a) with the complex of step b),
d) Detecting the analyte of interest by using the detectable signal of the label compound,
wherein the complex is a compound of formula I:

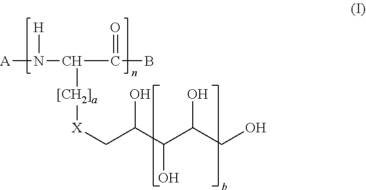

wherein A represents the label compound and B represents the analyte-specific binding agent or vice versa,
   X is selected from the group consisting of O, S, CH₂, SO, SO₂, 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15.

2. The method of the preceding aspect, wherein X=O, a=1 and b=0.

3. The method of any of the preceding aspects, wherein X=O, a=1 and b=1.

4. The method of any of the preceding aspects, wherein X=O, a=1 and b=2.

5. The method of any of the preceding aspects, wherein X=O, a=1 and b=3.

6. The method of any of the preceding aspects, wherein X=O, a=2 and b=0.

7. The method of any of the preceding aspects, wherein X=O, a=2 and b=1.

8. The method of any of the preceding aspects, wherein X=O, a=2 and b=2.

9. The method of any of the preceding aspects, wherein X=O, a=2 and b=3.

10. The method of any of the preceding aspects, wherein X=O, a=3 and b=0.

11. The method of any of the preceding aspects, wherein X=O, a=3 and b=1.

12. The method of any of the preceding aspects, wherein X=O, a=3 and b=2.

13. The method of any of the preceding aspects, wherein X=O, a=3 and b=3.

14. The method of any of the preceding aspects, wherein X=O, a=4 and b=0.

15. The method of any of the preceding aspects, wherein X=O, a=4 and b=1.

16. The method of any of the preceding aspects, wherein X=O, a=4 and b=2.

17. The method of any of the preceding aspects, wherein X=O, a=4 and b=3.

18. The method of any of the preceding aspects, wherein X=1,2,3-triazole, a=1 and b=0.

19. The method of any of the preceding aspects, wherein X=1,2,3-triazole, a=1 and b=1.

20. The method of any of the preceding aspects, wherein X=1,2,3-triazole, a=1 and b=2.

21. The method of any of the preceding aspects, wherein X=1,2,3-triazole, a=1 and b=3.

22. The method of any of the preceding aspects, wherein X=1,2,3-triazole, a=2 and b=0.

23. The method of any of the preceding aspects, wherein X=1,2,3-triazole, a=2 and b=1.

24. The method of any of the preceding aspects, wherein X=1,2,3-triazole, a=2 and b=2.

25. The method of any of the preceding aspects, wherein X=1,2,3-triazole, a=2 and b=3.

26. The method of any of the preceding aspects, wherein X=1,2,3-triazole, a=3 and b=0.

27. The method of any of the preceding aspects, wherein X=1,2,3-triazole, a=3 and b=1.

28. The method of any of the preceding aspects, wherein X=1,2,3-triazole, a=3 and b=2.

29. The method of any of the preceding aspects, wherein X=1,2,3-triazole, a=3 and b=3.

30. The method of any of the preceding aspects, wherein X=1,2,3-triazole, a=4 and b=0.

31. The method of any of the preceding aspects, wherein X=1,2,3-triazole, a=4 and b=1.

32. The method of any of the preceding aspects, wherein X=1,2,3-triazole, a=4 and b=2.

33. The method of any of the preceding aspects, wherein X=1,2,3-triazole, a=4 and b=3.

34. The method of any of the preceding aspects, wherein X=amide, a=1 and b=0.

35. The method of any of the preceding aspects, wherein X=amide, a=1 and b=1.

36. The method of any of the preceding aspects, wherein X=amide, a=1 and b=2.

37. The method of any of the preceding aspects, wherein X=amide, a=1 and b=3.

38. The method of any of the preceding aspects, wherein X=amide, a=2 and b=0.

39. The method of any of the preceding aspects, wherein X=amide, a=2 and b=1.

40. The method of any of the preceding aspects, wherein X=amide, a=2 and b=2.

41. The method of any of the preceding aspects, wherein X=amide, a=2 and b=3.

42. The method of any of the preceding aspects, wherein X=amide, a=3 and b=0.

43. The method of any of the preceding aspects, wherein X=amide, a=3 and b=1.

44. The method of any of the preceding aspects, wherein X=amide, a=3 and b=2.

45. The method of any of the preceding aspects, wherein X=amide, a=3 and b=3.

46. The method of any of the preceding aspects, wherein X=amide, a=4 and b=0.

47. The method of any of the preceding aspects, wherein X=amide, a=4 and b=1.

48. The method of any of the preceding aspects, wherein X=amide, a=4 and b=2.

49. The method of any of the preceding aspects, wherein X=amide, a=4 and b=3

50. The method of any of the preceding aspects, wherein a is 1.

51. The method of any of the preceding aspects, wherein a is 2.

52. The method of any of the preceding aspects, wherein a is 3.

53. The method of any of the preceding aspects, wherein a is 4.

54. The method of any of the preceding aspects, wherein the label compound is selected from the group consisting of an enzyme, a fluorescent dye, a luminescent dye, a metal chelate complex, and a moiety containing a radioisotope.

55. The method of any of the preceding aspects, wherein the label compound is capable of being induced to luminesce when electrochemically oxidized or reduced.

56. The method of any of the preceding aspects, wherein the label compound comprises a metal ion, which is Ru²⁺ or Ir³⁺.

57. The method of any of the preceding aspects, wherein the label compound is covalently bonded to the linker via a first conjugation method, wherein the first conjugation method is selected from the following group: click chemistry, amide, ester, imide, carbonate, carbamate, squarate, thiazole, thiazolidine, hydrazone, oxime, dihydropyridazine, thiol-maleimide, cycloaddition, photoclick, Staudinger ligation, Diels-Alder, tetrazine ligation, cross-coupling, Pictet-Spengler, quadricyclane.

58. The method of any of the preceding aspects, wherein the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein.

59. The method of any of the preceding aspects, wherein the analyte-specific binding agent is covalently bonded to the linker via a second conjugation method, wherein the second conjugation method is selected from the following group: click chemistry, amide, ester, imide, carbonate, carbamate, squarate, thiazole, thiazolidine, hydrazone, oxime, dihydropyridazine, thiol-maleimide, cycloaddition, tetrazine ligation, photoclick, Staudinger ligation, Diels-Alder, cross-coupling, Pictet-Spengler, quadricyclane.

60. The method of any of the preceding aspects, wherein A of formula I represents the label compound and B of formula I represents the analyte-specific binding agent.

61. The method of any of the preceding aspects, wherein B of formula I represents the label compound and A of formula I represents the analyte-specific binding agent.

62. The method of any of the preceding aspects, wherein prior, during or after step (c) the analyte is immobilized on a solid phase.

63. The method of any of the preceding aspects, wherein the sample is selected from the group consisting of sputum, saliva, liquor, urine, whole blood, hemolyzed whole blood, serum and plasma.

64. The method of any of the preceding aspects, wherein the complex of step (b) is provided in dissolved form, and step (c) is performed in a liquid aqueous buffer.

65. The method of any of the preceding aspects, wherein the liquid aqueous buffer is selected from phosphate, tris buffer, citrate, cacodylate, barbital, glycine, HEPES, MES, PIPES, MOPS, bis-tris methane, ADA, bis-tris propane, ACES, MOPSO, BES, AMPB, TES, DIPSO, MOBS, acetamidoglycine, TAPSO, TEA, POPSO, HEPPSO, EPS, HEPPS, tricine, gycinamide, Gly-Gly, HEPBS, bicine, TAPS and mixtures thereof.

66. The method of any of the preceding aspects, wherein the complex is selected from the groups consisting of at least one compound having at least one of the following formulae 4-I to 4-X:

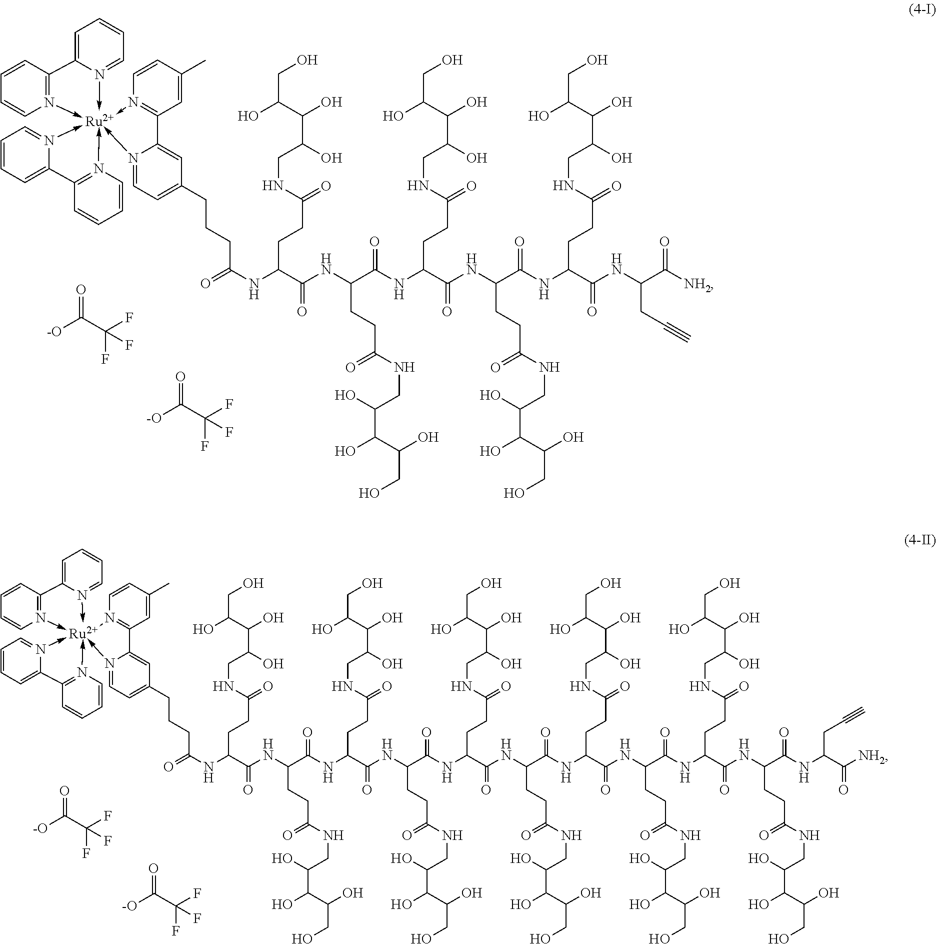

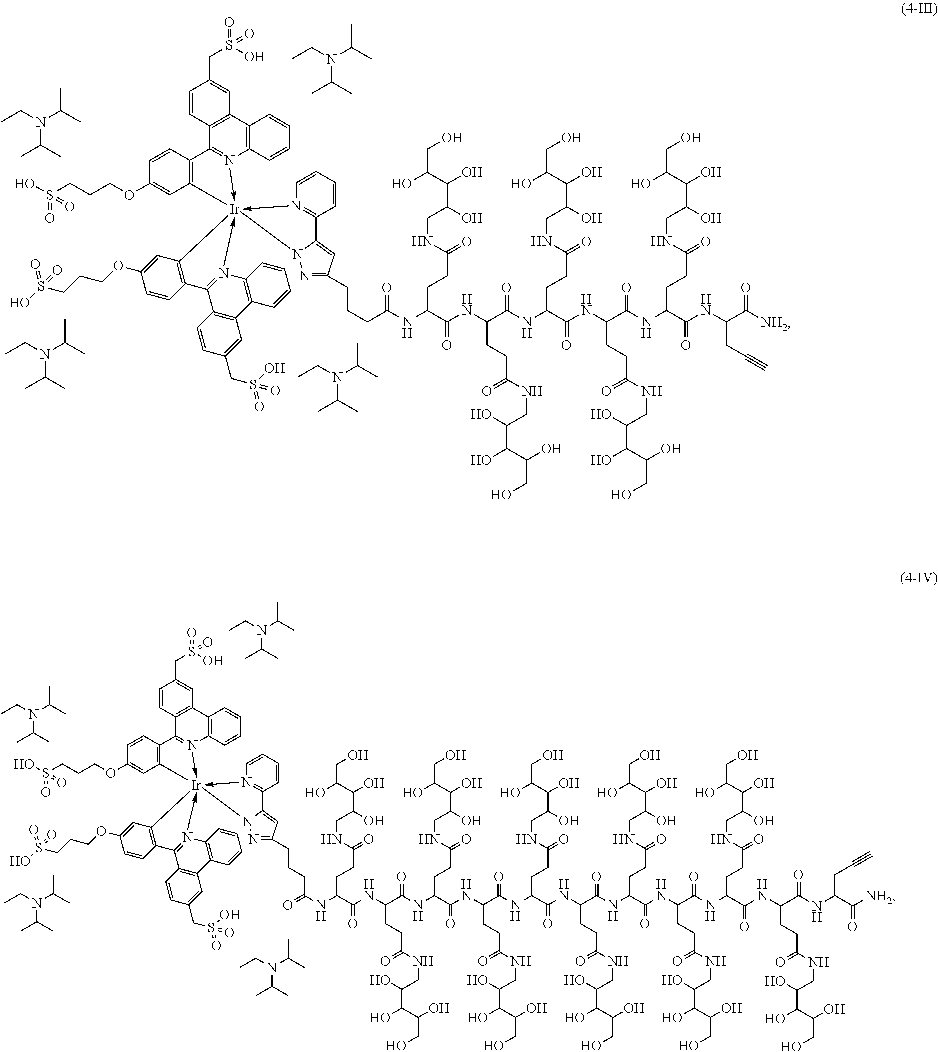

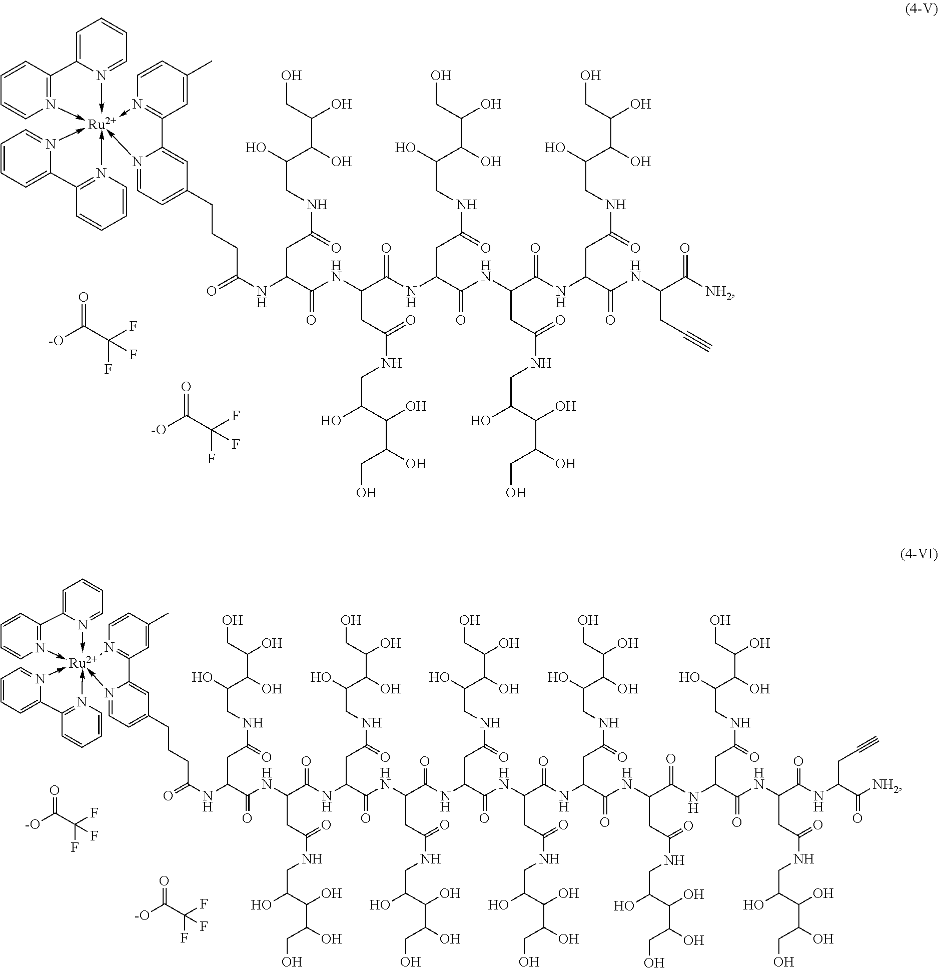

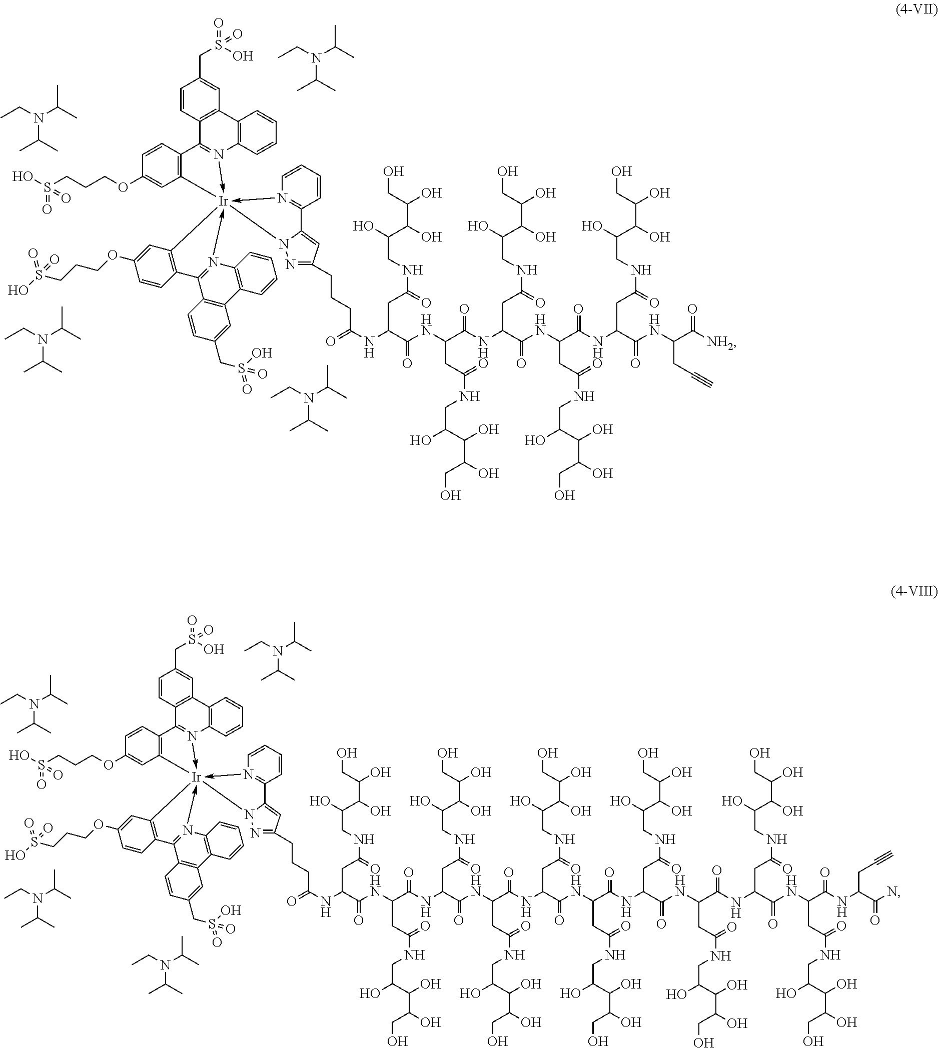

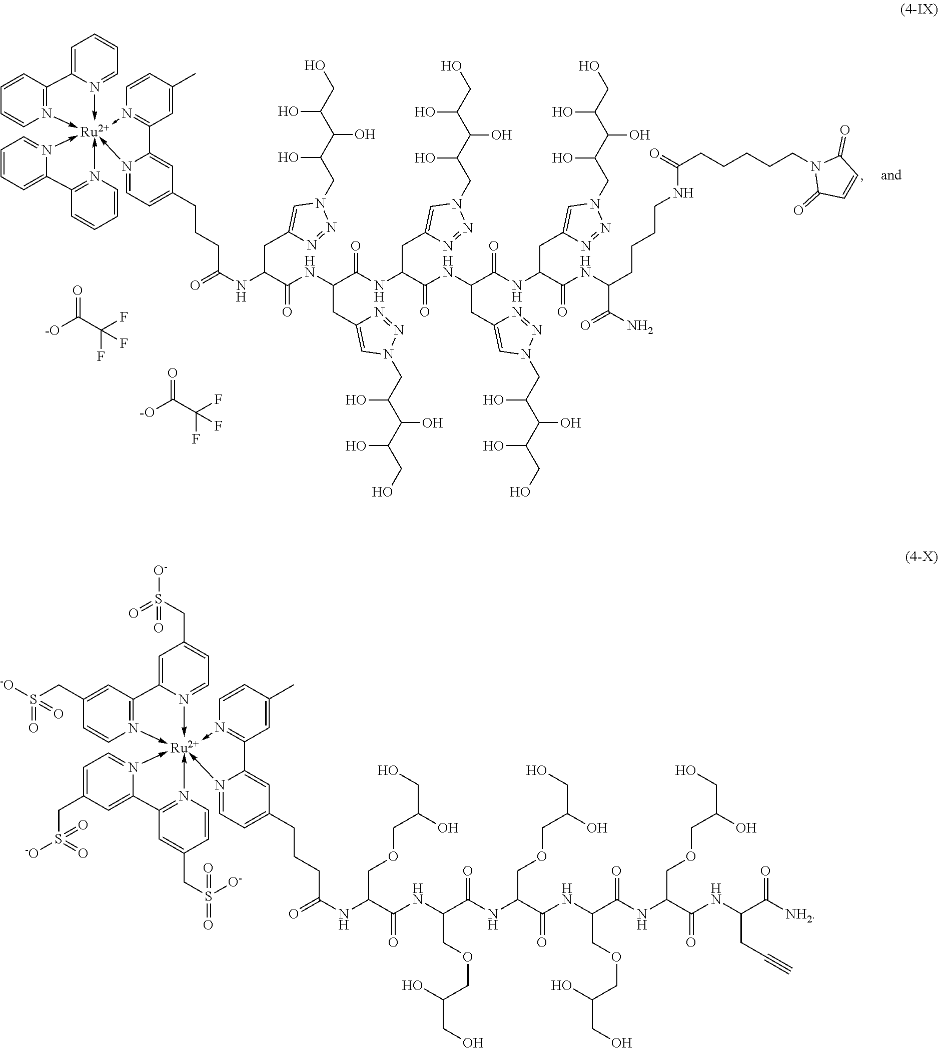

67. The method of any of the preceding aspects, wherein step b) comprises a peptide-based synthesis, preferably a solid phase peptide synthesis (SPPS).

68. Use of the method according to any of the proceeding aspects 1 to 67 for detecting the analyte of interest in the sample.

69. A kit for performing detection of an analyte of interest in a sample, the kit comprising in separate containers
a) a solid phase capable of immobilizing the analyte;
b) a compound of formula I:

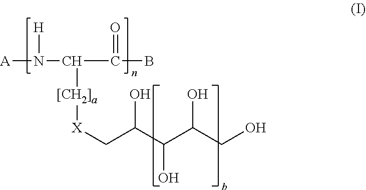

wherein A represents the label compound and B represents the analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15.

70. The kit of aspect 69, wherein the complex is embodied in dissolved form.

71. Use of the kit according to any of the proceeding claims 69 to 70 for detecting the analyte of interest in the sample.

72. A complex of formula I,

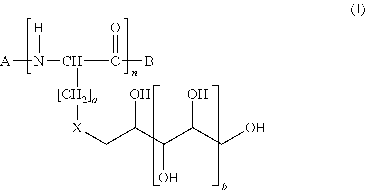

wherein A represents a label compound and B represents an analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15,
preferably wherein the compound is suitable to detect an analyte of interest in a sample.

73. The complex of aspect 72, wherein A or B is selected from the group consisting of a peptide, a polypeptide, and a protein.

74. The complex of any of aspects 72 to 73, wherein A comprises the analyte-specific binding agent and B comprises the label compound, or B comprises the analyte-specific binding agent and A comprises the label compound.

75. The complex of any of aspects 72 to 74, wherein the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein, and the label compound is selected from the group consisting of enzyme, a fluorescent dye, a luminescent dye, a metal chelate complex, and a moiety containing a radioisotope.

76. A method to synthesize a complex of any of aspects 72 to 75 comprising the steps of
a) Providing a monomer or derivatives thereof, wherein the monomer is an amino acid comprising an amino group, a carboxy group and at least one hydroxyl group or at least two hydroxyl groups, wherein the amino group or the carboxy group is protected by a first protecting group, and the at least one or each hydroxyl group is protected by a second protecting group,
b) Using the monomer in a process of solid phase peptide synthesis, cleaving the first and second protection group and forming a complex of formula I

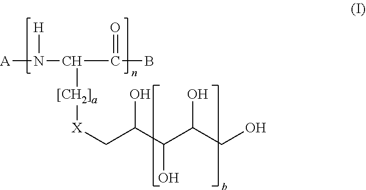

wherein A represents the label compound and B represents the analyte-specific binding agent or vice versa,
   X is selected from the group consisting of O, S, CH₂, SO, SO₂ or 1,2,3-triazole, and amide,
   a is 1 to 5, preferably 1 to 4, more preferably 1 to 2,
   b is 0 to 3, and
   n is an integer from 1 to 20, preferably 1 to 15, or from 2 to 20, preferably 2 to 15.

77. The method of aspect 76, wherein the first and/or second protecting group is selected from the group consisting of Fmoc, tBu, Boc, ether, ester and acetal.

78. The method of any of aspects 76 to 77, wherein a monomer or a derivative thereof is selected from the following formulae m-1 to m-3:

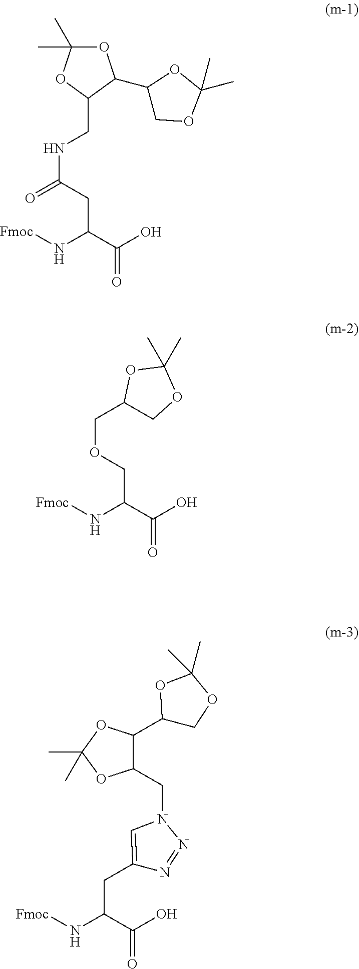

wherein Fmoc means a 9-fluorenylmethoxycarbonyl protecting group.

79. A monomer used for a peptide-based synthesis comprising the following formula II

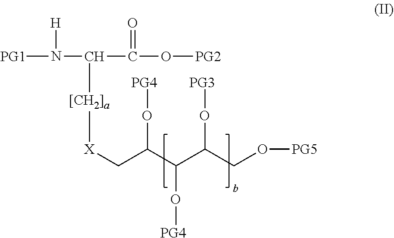

wherein PG1, PG2, PG3, PG4, PG5, PG6 are each independently a protecting group, preferably if PG1 is a protecting group, PG2=H or vice versa,
wherein X is selected from the group consisting of O, S, CH₂, SO, SO₂ or 1,2,3-triazole, and amide,
wherein a is 1 to 5, preferably 1 to 4, more preferably 1 to 2,
wherein b is 0 to 3.

80. The monomer of aspect 79, wherein the protecting groups PG1, PG2, PG3, PG4, PG5, PG6 are independently selected from the group consisting of Fmoc, tBu, Boc, ether, ester and acetal.

81. The monomer of any of the preceding aspects 79 to 80, wherein at least two of the protecting groups selecting from the group consisting of PG1, PG2, PG3, PG4, PG5, PG6 are linked to each other, for example by at least one covalent bond.

82. The monomer of any of the preceding aspects 79 to 81, wherein a monomer or a derivative thereof is selected from the following formulae m-1 to m-3:

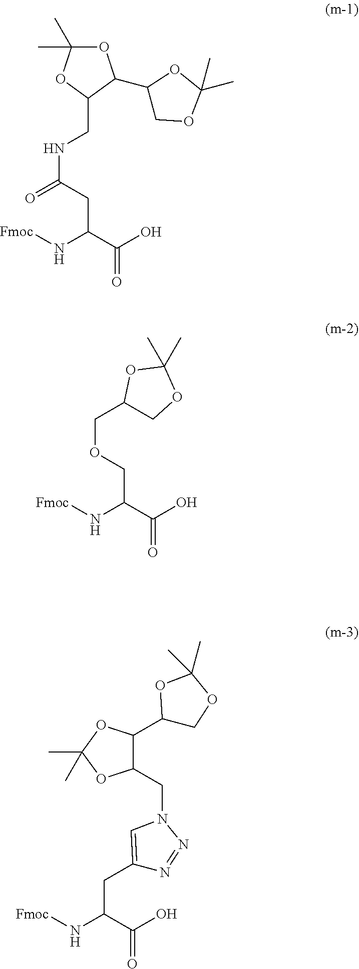

### EXAMPLES

The following examples are provided to illustrate, but not to limit the presently claimed invention.

### Example 1: Synthesis of the “Clicked” Side Chain Derivative

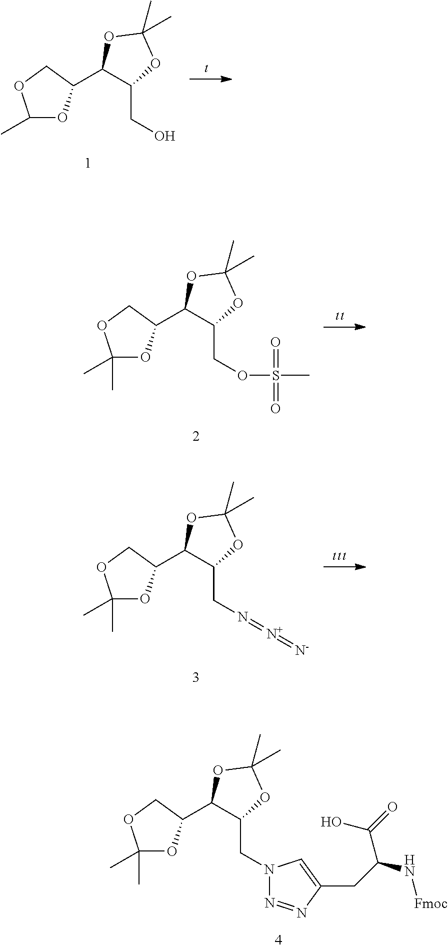

Scheme 1. Synthesis of compound 4. i) MsCl, DMAP (cat.), dry pyridine, 1.5 h; ii) NaN3, DMF, 6 h; iii) Fmoc-L-propargylglycine, CuSO4, sodium-L-ascorbate, DMF/H2O, 3 h.

### Synthesis of Compound 2

2,3:4,5-di-O-Isopropylidene-D-arabitol (267 mg, 1.19 mmol) was dissolved in dry pyridine (3 mL) and cooled in an ice bath. 4-(Dimethylamino)pyridine (cat.) and methanesulfonyl chloride (110 μl, 1.43 mmol) were added. The reaction mixture was stirred 1.5 h at 0° C. Pyridine was evaporated in vacuo, the residue was dissolved in CH₂Cl₂ and washed with H₂O and NaCl saturated solution. The organic phase was dried over anhydrous Na₂SO₄, the solvent was evaporated in vacuo and the oily residue was purified via flash-column chromatography (SiO₂, n-hexane:EtOAc 7:3) to give 318 mg of mesylate 2 (86%); 1H NMR (400 MHZ, CHLOROFORM-d) δ ppm 1.32 (s, 3H) 1.36-1.43 (m, 9H) 3.01-3.12 (s, 3H) 3.66-3.75 (m, 1H) 3.91-3.97 (m, 1H) 4.00-4.07 (m, 1H) 4.11-4.20 (m, 2H) 4.26-4.33 (m, 1H) 4.47-4.58 (m, 1H).

### Synthesis of Compound 3

Mesylate 2 (1.29 g, 4.16 mmol) was dissolved in DMF (20 mL), NaN₃ (324 mg, 4.99 mmol) was added and the reaction mixture was stirred at 85° C. for 6 h. The solvent was evaporated in vacuo, the residue was dissolved in EtOAc and washed with H₂O and NaCl saturated solution. The organic phase was dried over anhydrous Na₂SO₄, the solvent was evaporated in vacuo and the residue was purified via flash-column chromatography (SiO₂, n-hexane:EtOAc 9:1) to give 788 mg of azide 3 (74%); 1H NMR (400 MHZ, CHLOROFORM-d) 8 ppm 1.32 (s, 3H) 1.38 (m, 6H) 1.44 (s, 3H) 3.31 (m, 1H) 3.65 (m, 1H) 3.71-3.78 (m, 1H) 3.91-3.98 (m, 1H) 3.98-4.04 (m, 1H) 4.06-4.17 (m, 2H).

### Synthesis of Compound 4

Azide 3 (1.63 g, 6.34 mmol) and Fmoc-L-propargylglycine (2.13 g, 6.34 mmol) were dissolved in 25 mL DMF and 0.5 mL H₂O. CuSO₄ (253 mg, 1.58 mmol) and sodium-L-ascorbate (3.13 g, 18.8 mmol) were added and the reaction mixture was stirred at r.t. for 3 h. H₂O was added and the mixture was extracted 3 times with Et₂O. The organic phase was dried over anhydrous Na₂SO₄, the solvent was evaporated in vacuo and the residue was purified via chromatography (RP-C18AQ, H₂O:ACN gradient elution from 9:1 to 1:9) to give 2.14 g of the Fmoc protected amino acid 4 (57%); 1H NMR (400 MHZ, CHLOROFORM-d) 8 ppm 1.25-1.52 (m, 12H) 3.16-3.38 (m, 1H) 3.38-3.50 (m, 1H) 3.50-3.61 (m, 1H) 3.91-3.98 (m, 1H) 4.00-4.08 (m, 1H) 4.10-4.17 (m, 1H) 4.19-4.28 (m, 2H) 4.31-4.46 (m, 3H) 4.48-4.68 (m, 1H) 4.69-4.95 (m, 1H) 5.78-6.15 (m, 1H) 7.27-7.34 (m, 2H) 7.35-7.44 (m, 2H) 7.53-7.63 (m, 3H) 7.70-7.87 (m, 2H).

### Example 2: Synthesis of Asp/Glu Derivatives

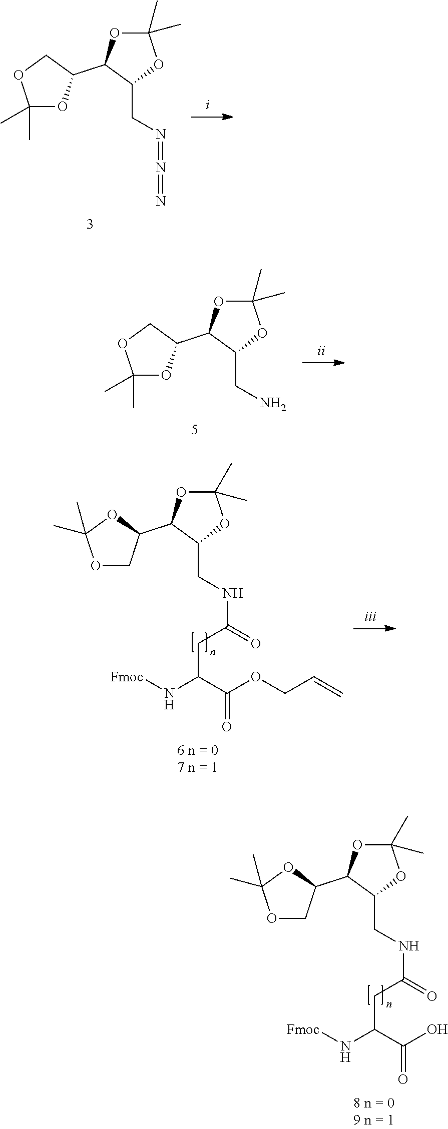

Scheme 2. Synthesis of Asp/Glu derivatives. i) H2, Pd/C (cat.), methanol, 8 h; ii) Fmoc-Asp-OAll or Glu-Asp-OAll, HBTU, DIPEA, dry DMF, 14 h; iii) Pd(PPh3) 4, morpholine, dry THF, 30 min.

### Synthesis of Compound 5

Azide 3 (2.19 g, 8.5 mmol) was dissolved in methanol (30 mL) and then palladium (0) on activated charcoal was added (cat.). After evacuating the air and saturating the atmosphere with H₂, the reaction mixture was vigorously stirred for 8 h under constant supply of H₂. The catalyst was removed by filtration through a pad of celite and the filtrate was dried at reduced pressure obtaining 1.95 g of amine 5, which was directly used for the following step without further purification (98%); 1H NMR (400 MHz, CHLOROFORM-d) δ ppm 1.27 (s, 3H) 1.30 (s, 3H) 1.31-1.35 (m, 6H) 2.77 (dd, J=13.4, 6.4 Hz, 1H) 2.97 (dd, J=13.4, 3.6 Hz, 1H) 3.55 (t, J=8.0 Hz, 1H) 3.82-3.92 (m, 2H) 3.93-4.01 (m, 1H) 4.02-4.11 (m, 1H).

### Synthesis of Compound 6

In a 3-neck round-bottom flask under Argon atmosphere Fmoc-Asp-OAll (2.28 g, 5.76 mmol) was dissolved in dry DMF (7 mL) and after that (2-(1H-benzotriazol-1-yl)-1,1,3,3-tetramethyluronium hexafluorophosphate (HBTU, 2.4 g, 6.34 mmol) and diisopropylethylamine (DIPEA, 1.54 mL, 8.64 mmol) were added. After stirring for 10 minutes at r.t. amine 2 (1.47 g, 6.34 mmol), previously dissolved dry DMF (3 mL), was added and the mixture was let stirring 15 h at r.t. The reaction mixture was poured in H₂O and the aqueous phase was extracted 3 times with EtOAc. The combined organic layer was dried over anhydrous Na₂SO₄ and concentrated at reduced pressure. The residue was then purified twice by flash-column chromatography (SiO₂, n-hexane/EtOAc gradient elution from 3:2 to 2:3) and (SiO₂, n-hexane/EtOAc gradient elution from 3:2 to 1:4) obtaining 2.88 g of product (82%); ¹H NMR (400 MHZ, CHLOROFORM-d) δ ppm 1.28-1.40 (m, 9H) 1.42 (s, 3H) 2.74 (dd, J=15.9, 4.4 Hz, 1H) 2.98 (dd, J=15.8, 4.4 Hz, 1H) 3.48-3.57 (m, 3H) 3.89-4.03 (m, 3H) 4.07-4.18 (m, 1H) 4.18-4.34 (m, 2H) 4.37-4.52 (m, 1H) 4.52-4.77 (m, 3H) 5.23 (dd, J=10.4, 1.1 Hz, 1H) 5.32 (br d, J=17.2 Hz, 1H) 5.83-5.97 (m, 1H) 6.03-6.30 (m, 2H) 7.27-7.34 (m, 2H) 7.34-7.44 (m, 2H) 7.52-7.66 (m, 2H) 7.75 (d, J=7.5 Hz, 2H).

### Synthesis of Compound 7

The allyloxycarbonyl-protected Glu derivative 7 was prepared using the same procedure as for compound 6 starting from Fmoc-Glu-OAll (3.19 g, 7.81 mmol), HBTU (3.26 g, 8.6 mmol), DIPEA (2 mL, 11.7 mmol) and amine 5 (1.987 g, 8.6 mmol). After the work up, purification was performed by flash-column chromatography (SiO₂, n-hexane/EtOAc gradient elution from 1:1 to 1:4) obtaining 4.75 g of the desired product (97%); 1H NMR (400 MHZ, CHLOROFORM-d) 8 ppm 1.30-1.38 (m, 9H) 1.41 (s, 3H) 1.95-2.35 (m, 4H) 3.49-3.59 (m, 3H) 3.91-4.05 (m, 3H) 4.13 (dd, J=8.5, 6.1 Hz, 1H) 4.18-4.25 (m, 1H) 4.31-4.46 (m, 3H) 4.64 (d, J=5.6 Hz, 2H) 5.25 (dd, J=10.4, 1.1 Hz, 1H) 5.32 (d, J=17.2 Hz, 1H) 5.74 (d, J=7.4 Hz, 1H) 5.83-5.96 (m, 1H) 6.18-6.28 (m, 1H) 7.31 (tt, J=7.4, 1.1 Hz, 2H) 7.39 (t, J=7.5 Hz, 2H) 7.59 (t, J=6.3 Hz, 2H) 7.76 (d, J=7.1 Hz, 2H).

### Synthesis of Compound 8

In a three-neck round bottom flask under Argon atmosphere compound 6 (2.46 g, 4.04 mmol) and tetrakis(triphenylphosphine) palladium (0) (462 mg, 0.40 mmol) were dissolved in dry THF (60 mL) and after that morpholine (0.524 mL, 6.06 mmol) was added. The mixture was stirred for 30 min after which 20 mL H₂O and 20 mL NaHCO₃ saturated solution were added. The formed precipitate was removed by filtration and the filtrate was acidified to pH ˜ 1-2 using 2N HCl. The mixture was then extracted 3 times with EtOAc and the organic layer was dried on anhydrous Na₂SO₄ and concentrated at reduced pressure. The residue was purified twice by flash-column chromatography (RP-C18AQ, H2O/ACN gradient elution from 3:2 to 2:3) and (RP-C18AQ, H2O/ACN gradient elution from 3:2 to 3:7) obtaining 1.60 g of product (70%); 1H NMR (400 MHZ, CHLOROFORM-d) 8 ppm 1.29-1.51 (m, 12H) 2.75-2.87 (m, 1H) 2.94-3.08 (m, 1H) 3.48-3.65 (m, 3H) 3.92-4.06 (m, 2H) 4.07-4.17 (m, 2H) 4.18-4.25 (m, 1H) 4.28-4.43 (m, 2H) 4.49-4.66 (m, 1H) 6.19-6.37 (m, 1H) 6.67-6.84 (m, 1H) 7.25-7.34 (m, 2H) 7.33-7.45 (m, 2H) 7.52-7.67 (m, 2H) 7.70-7.81 (m, 2H).

### Synthesis of Compound 9

The deprotected Glu derivative 9 was prepared using the same procedure as for compound 8 starting from the protected compound 7 (4.62 g, 7.42 mmol), tetrakis (triphenylphosphine) palladium (0) (429 mg, 0.37 mmol) and morpholine (0.770 mL, 8.90 mmol). After the work up the raw product was purified by flash column chromatography (RP-C18AQ, H2O/ACN gradient elution from 3:2 to 2:3) obtaining 3.64 g of product (84%); 1H NMR (400 MHz, CHLOROFORM-d) δ ppm 1.31-1.41 (m, 9H) 1.42-1.48 (m, 3H) 2.06-2.16 (m, 1H) 2.17-2.30 (m, 1H) 2.38-2.49 (m, 1H) 2.50-2.63 (m, 1H) 3.50-3.65 (m, 3H) 3.94-4.08 (m, 3H) 4.13-4.19 (m, 1H) 4.20-4.26 (m, 1H) 4.30-4.48 (m, 3H) 6.04 (br d, J=6.8 Hz, 1H) 6.42-6.57 (m, 1H) 7.32 (td, J=7.4, 0.8 Hz, 2H) 7.41 (t, J=7.5 Hz, 2H) 7.60 (t, J=6.3 Hz, 2H) 7.77 (d, J=7.5 Hz, 2H).

### Example 3: Synthesis of Ser Derivative

Example 3 shows the synthesis of Ser derivative (Scheme 3).

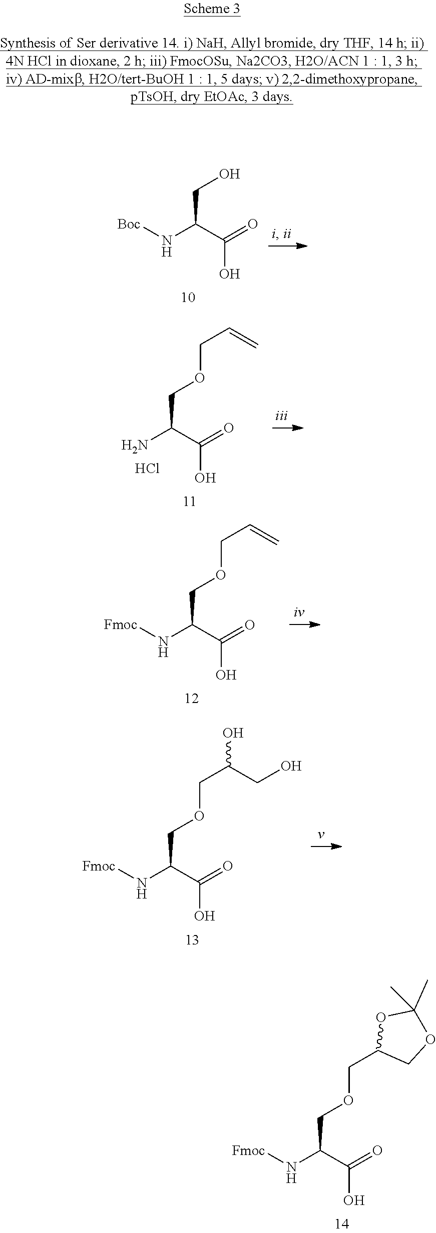

### Synthesis of Compound 11

Under Ar atmosphere NaH (60% dispersion in mineral oil, 1.46 g, 36.5 mmol) was added in portions to dry THF (20 mL), previously cooled at 0° C. After stirring for 5 min Boc-L-Serine (10, 2.5 g, 12.2 mmol) was slowly added over a period of 30 min. After completion of the addition, the mixture was let stirring for additional 30 min and next allyl bromide (1.84 g, 15.25 mmol) was added. The reaction mixture was stirred overnight (14 h), slowly heating to r.t. The reaction was quenched by adding 5 mL of H₂O and the solvent was evaporated at reduced pressure. The residue was dissolved in H₂O and, after cooling it in an ice bath, the solution was acidified to pH ˜ 1-2 using 2N HCl. The aqueous phase was extracted 5 times with EtOAc, the organic layer was dried over anhydrous Na₂SO₄ and dried in vacuo. After dissolving the residue in CH₂Cl₂ (10 mL), 5 mL of 4N HCl in 1,4-dioxane were added and the mixture was stirred for 2 h at r.t. The solvent was evaporated at reduced pressure and the residue was treated with Et₂O, obtaining a white precipitate which was filtered off and dried in vacuo. 1.92 g of product were obtained as a hydrochloride salt (87%); 1H NMR (400 MHZ, DEUTERIUM OXIDE) 8 ppm 3.66-3.78 (m, 1H) 3.79-4.04 (m, 3H) 4.04-4.32 (m, 1H) 4.08-4.20 (m, 1H) 5.06-5.32 (m, 2H) 5.64-5.87 (m, 1H).

### Synthesis of Compound 12

The allyl Ser derivative 11 (1.79 g, 9.92 mmol) was dissolved in a H₂O/ACN 1:1 mixture (30 mL) and after that Na₂CO₃ (2.10 g, 19.8 mmol) and Fmoc-N-hydroxysuccinimide ester (FmocOSu, 3.68 g, 10.9 mmol) were added. The reaction mixture was stirred for 3 h at r.t. and then the organic solvent was evaporated at reduced pressure. The pH was adjusted to 1-2 with 6N HCl and the aqueous layer was extracted 5 times with EtOAc. The organic phase was then dried over anhydrous Na₂SO₄ and concentrated at reduced pressure. The residue was purified by flash-column chromatography (RP-C18AQ, H₂O/ACN gradient elution from 3:2 to 2:3). The fractions containing the product were pooled and the organic solvent was evaporated at reduced pressure. The aqueous phase was then extracted with EtOAc and the organic layer was dried in vacuo, giving 3.63 g of the Fmoc-protected derivative 12 (96%); 1H NMR (400 MHZ, CHLOROFORM-d) 8 ppm 3.60-3.79 (m, 1H) 3.87-4.16 (m, 3H) 4.24 (br t, J=7.1 Hz, 1H) 4.32-4.61 (m, 3H) 5.21 (d, J=10.3 Hz, 1H) 5.27 (d, J=17.2 Hz, 1H) 5.63-5.77 (m, 1H) 5.77-5.93 (m, 1H) 7.27-7.34 (m, 2H) 7.35-7.43 (m, 2H) 7.51-7.65 (m, 2H) 7.68-7.80 (m, 2H).

### Synthesis of Compound 13

Fmoc-Ser derivative 12 (3.63 g, 9.90 mmol), asymmetric dihydroxylation mix β (AD-mix β, 17.86 g) and Na₂CO₃ (1.05 g, 9.90 mmol) were suspended in a H₂O/tert-BuOH 1:1 mixture (60 mL) and the reaction mixture was stirred at 4° C. for 5 days. The OsO₄ present in the AD-mix β was neutralized by adding 4 g of Na₂S₂O₃ and stirring for 10 min at r.t. After diluting with 100 mL H₂O, 30 mL EtOAc were added and the 2 phases formed were separated. The pH of the aqueous phase was adjusted to 1-2 units using 6N HCl and the aqueous layer was extracted 4 times with EtOAc. The combined organic layer was dried over anhydrous Na₂SO₄ and concentrated at reduced pressure. The residue was finally purified by flash-column chromatography (RP-C18AQ, H₂O/ACN gradient elution from 3:1 to 1:1) obtaining 2.24 g of the vicinal diol 13 (56%); 1H NMR (400 MHZ, acetone) 8 ppm 3.31 (s, 2H) 3.47-3.63 (m, 4H) 3.71-3.86 (m, 2H) 3.91-4.03 (m, 1H) 4.22-4.30 (m, 1H) 4.30-4.39 (m, 2H) 4.40-4.52 (m, 1H) 6.85 (d, J=8.4 Hz, 1H) 7.29-7.36 (m, 2H) 7.37-7.44 (m, 2H) 7.64-7.75 (m, 2H) 7.86 (d, J=7.5 Hz, 2H).

### Synthesis of Compound 14

Diol derivative 13 (2.19 g, 5.46 mmol) was suspended in dry EtOAc (30 mL) and after that 2,2-dimethoxypropane (27 mL, 218.3 mmol) was added. Finally p-toluenesulfonic acid (105 mg, 0.55 mmol) was added and the reaction mixture was stirred at r.t. for 3 days. After adding 30 mL of a 5% NaHCO₃ solution the organic solvent was evaporated at reduced pressure and the aqueous phase was directly injected for chromatographic separation (RP-C18AQ, H₂O/ACN gradient elution from 4:1 to 1:1). The fractions containing the product were pooled and the organic solvent was removed at reduced pressure. After cooling the residual solution in an ice bath, the pH was adjusted to 1-2 units using 1N HCl and the resulting mixture was extracted 3 times with EtOAc. The organic phase was dried over anhydrous Na₂SO₄ and concentrated in vacuo, affording 1.96 g of the acetal-protected product 14 (81%); 1H NMR (400 MHZ, CHLOROFORM-d) 8 ppm 1.29-1.50 (m, 6H) 3.43-3.60 (m, 2H) 3.65-3.83 (m, 2H) 3.94-4.07 (m, 2H) 4.17-4.30 (m, 2H) 4.31-4.48 (m, 2H) 4.50-4.62 (m, 1H) 5.79-5.91 (m, 1H) 6.04 (br s, 1H) 7.26-7.34 (m, 2H) 7.35-7.44 (m, 2H) 7.54 (br s, 1H) 7.75 (d, J=7.5 Hz, 2H).

### Example 4: Synthesis of Hexahydroxy Glu Derivative

Example 4 Shows the Synthesis of Hexahydroxy Glu Derivative (Scheme 4).

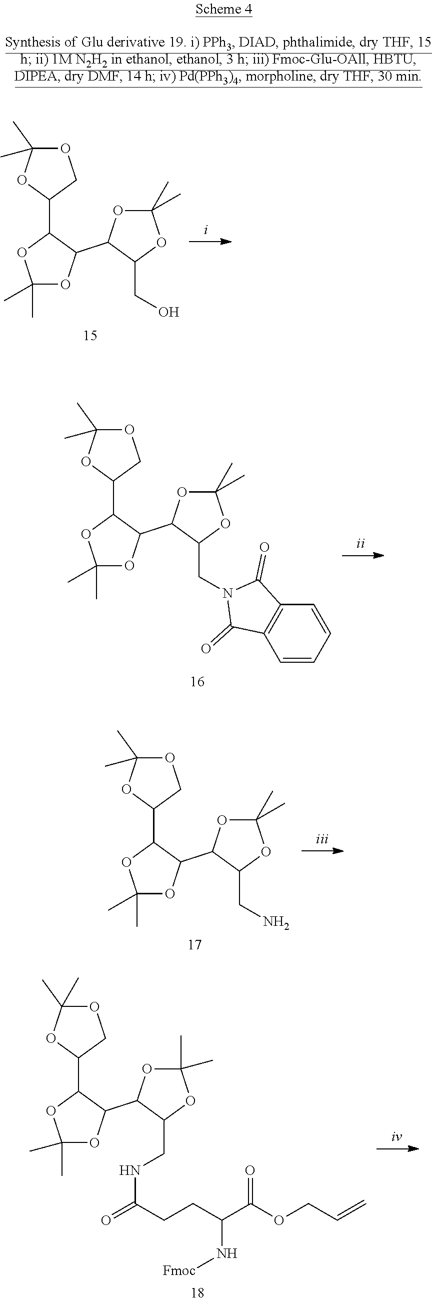

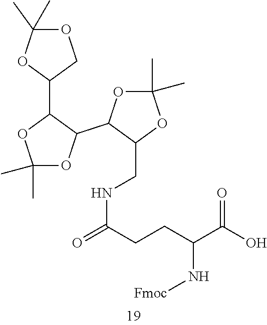

### Synthesis of Compound 16

Tri-isopropylidene heptol (15, 1.08 g, 3.25 mmol) was dissolved in dry THF (12 mL) and, after cooling the solution at −15° C., triphenylphosphine (1.11 g, 4.22 mmol) and phthtalimide (621 mg, 4.22 mmol) were added. Finally, diisopropyl azodicarboxylate (DIAD, 0.831 mL, 4.22 mmol) was added dropwise over 5 minutes and, after completion of the addition, the mixture was let slowly heating to r.t. and stirred for 15 h. After evaporating the solvent at reduced pressure, the residue was treated with 2N NaOH and the obtained mixture was extracted 3 times with EtOAc. The combined organic layer was concentrated at reduced pressure and the raw product was purified by flash-column chromatography (SiO₂, n-hexane/EtOAc gradient elution from 4:1 to 2:3), obtaining 1.31 g of product 16 (87%); ¹H NMR (400 MHZ, CHLOROFORM-d) 8 ppm 1.25 (s, 3H) 1.31 (s, 3H) 1.36 (s, 3H) 1.39 (s, 3H) 1.43 (s, 3H) 1.51 (s, 3H) 3.68 (dd, J=14.2, 2.9 Hz, 1H) 3.94-4.14 (m, 5H) 4.18 (dd, J=14.1, 10.2 Hz, 1H) 4.33 (dd, J=6.5, 2.0 Hz, 1H) 4.54-4.61 (m, 1H) 7.63-7.71 (m, 1H) 7.76-7.84 (m, 1H).

### Synthesis of Compound 17

Phthalimido derivative 16 (1.76 g, 3.82 mmol) was dissolved in ethanol (30 mL) and after that 1 M hydrazine in ethanol (7.64 mL, 7.64 mmol) was added. The reaction mixture was then heated to reflux temperature and stirred for 3 h. After cooling down to r.t., the abundant white precipitate was removed by filtration and the filtrate was concentrated at reduced pressure. The obtained residue was suspended in IM KOH solution (30 mL) and the aqueous phase was extracted 5 times with EtOAc. The combined organic layer was dried over anhydrous Na₂SO₄ and concentrated at reduced pressure. Finally, the raw product was purified by flash-column chromatography (SiO₂, gradient elution from EtOAc/methanol 98:2+1% Et₃N to EtOAc/methanol 9:1+1% Et₃N) obtaining 1.08 g of amine 17 (86%). 1H NMR (400 MHz, CHLOROFORM-d) δ ppm 1.26-1.34 (m, 9H) 1.35-1.40 (m, 6H) 1.42 (br s, 2H) 1.47 (s, 3H) 2.87 (dd, J=13.3, 3.9 Hz, 1H) 3.03 (dd, J=13.2, 8.1 Hz, 1H) 3.82 (dd, J=7.7, 1.5 Hz, 1H) 3.86-3.95 (m, 2H) 3.95-4.02 (m, 1H) 4.06-4.12 (m, 1H) 4.13-4.20 (m, 1H) 4.25 (dd, J=6.8, 1.6 Hz, 1H).

### Synthesis of Compound 18

In a 3-neck round-bottom flask under Argon atmosphere Fmoc-Glu-OAll (2.12 g, 5.19 mmol) was dissolved in dry DMF (5 mL) and after that HBTU (2.16 g, 5.71 mmol) and DIPEA (1.36 mL, 7.78 mmol) were added. After stirring for 10 minutes at r.t., amine 17 (1.89 g, 5.71 mmol), previously dissolved dry DMF (5 mL), was added and the mixture was let stirring for 15 h at r.t. The reaction mixture was poured in H₂O and the aqueous phase was extracted 3 times with EtOAc. The combined organic layer was dried over anhydrous Na₂SO₄ and concentrated at reduced pressure. The residue was then purified twice by flash-column chromatography (SiO₂, n-hexane/EtOAc gradient elution from 1:1 to 1:4). The fractions containing impurities were pooled and purified again by flash-column chromatography (SiO₂, n-hexane/EtOAc gradient elution from 3:2 to 1:4). 3.22 g of product were obtained (86%); 1H NMR (400 MHZ, CHLOROFORM-d) 8 ppm 1.27-1.36 (m, 9H) 1.36-1.41 (m, 6H) 1.43-1.51 (m, 3H) 2.15-2.30 (m, 3H) 3.24-3.36 (m, 1H) 3.81-4.02 (m, 5H) 4.05-4.13 (m, 2H) 4.16-4.30 (m, 3H) 4.32-4.44 (m, 3H) 4.62 (d, J=5.5 Hz, 2H) 5.22 (d, J=10.4 Hz, 1H) 5.31 (d, J=17.3 Hz, 1H) 5.77-5.96 (m, 2H) 6.19-6.32 (m, 1H) 7.26-7.33 (m, 2H) 7.34-7.42 (m, 2H) 7.51-7.64 (m, 2H) 7.70-7.78 (m, 2H).

### Synthesis of Compound 19

In a 3-neck round-bottom flask under Argon atmosphere the allyloxycarbonyl-protected derivative 18 (3.16 g, 4.37 mmol) and tetrakis(triphenylphosphine) palladium (0) (252 mg, 0.22 mmol) were dissolved in dry THF (30 mL morpholine (0.454 mL, 5.24 mmol) was added and the mixture was then stirred for 20 min at r.t. After adding 5% NaHCO₃ solution (20 mL), the formed precipitate was removed by filtration and the filtrate was acidified to pH ˜ 1-2 using 2N HCl. The mixture was then extracted 3 times with EtOAc and the organic layer was dried on anhydrous Na₂SO₄ and concentrated at reduced pressure. The residue was f purified by flash-column chromatography (RP-C18AQ, H₂O/ACN gradient elution from 3:2 to 2:3). The fractions containing the product were pooled and the organic solvent was evaporated at reduced pressure. Finally, the remaining aqueous layer was extracted 3 times with EtOAc and the organic layer was dried over anhydrous Na₂SO₄ and concentrated to dryness, giving 2.56 g of the deprotected product 19 (86%); 1H NMR (400 MHZ, ACETONITRILE-d₃) δ ppm 1.27-1.32 (m, 6H) 1.32-1.40 (m, 9H) 1.45 (s, 3H) 1.85-1.94 (m, 1H) 2.01-2.20 (m, 2H) 2.25-2.38 (m, 2H) 3.24-3.36 (m, 1H) 3.60-3.72 (m, 1H) 3.81-3.93 (m, 2H) 3.92-4.01 (m, 1H) 4.03-4.13 (m, 2H) 4.11-4.20 (m, 1H) 4.21-4.40 (m, 5H) 6.37 (d, J=7.7 Hz, 1H) 6.72 (br s, 1H) 7.29-7.40 (m, 2H) 7.39-7.49 (m, 2H) 7.69 (m, J=7.2, 3.90 Hz, 2H) 7.85 (d, J=7.5 Hz, 2H).

### Example 5: Peptide Synthesis

Peptides were synthesized by means of fluorenylmethyloxycarbonyl (Fmoc) solid phase peptide synthesis on a multiple peptide synthesizer e.g. from Multisyntech. For this 4.0 equivalents of each amino acid derivative were used. Amino acid derivatives were dissolved in N-methylpyrrolidone containing 1 equivalent of 1-Hydroxy-7-azabenzotriazol. Peptides were synthesized on Tentagel R resin. Coupling reactions were carried out for 5 minutes in dimethylformamide as a reaction medium with 4 equivalents HATU and 8 equivalents of N,N-Diisopropylethylamine (DIPEA) relative to resin loading. The Fmoc group was cleaved in 8 minutes after each synthesis step using 25% piperidine in dimethylformamide. In case of labels presenting an alkyne functionality, the alkyne group was introduced by coupling of Fmoc-propargylglycine. In case of labels containing a maleimido-functionalized lysine, the lysine was introduced by coupling of Fmoc-ivDde lysine. After the synthesis the resin was treated with a solution of 2% hydrazine in DMF for 2×30 min to liberate ivDde-protected lysine. Afterwards 6-Maleimidohexanoic acid N-hydroxysuccinimide ester (10 eq.) and DIPEA (10 eq.) were added to the resin and incubated for 1 h followed by 3 washing steps with DMF. Release of the peptide from the synthesis resin and cleavage of the acid-labile protecting groups was achieved in 3 hours at room temperature with a cocktail containing trifluoroacetic acid, triisopropylsilane, and water (38:1:1). The reaction solution was subsequently mixed with cooled diisopropyl ether to precipitate the peptide. The precipitate was filtered, washed again with cold diisopropyl ether, dissolved in a small amount of aqueous acetic acid and lyophilized. The crude material obtained was purified by preparative RP-HPLC using a gradient of acetonitrile/water containing 0.1% trifluoroacetic acid. The identity of the purified material was checked by means of ion spray mass spectrometry.

Release of the peptide from the synthesis resin and the cleavage of the acid-labile protecting groups was achieved in 3 hours at room temperature with a cocktail containing trifluoroacetic acid, triisopropylsilane, and water (38:1:1). The reaction solution was subsequently mixed with cooled diisopropyl ether to precipitate the peptide. The precipitate was filtered, washed again with cold diisopropyl ether, dissolved in a small amount of aqueous acetic acid and lyophilized. The crude material obtained was purified by preparative RP-HPLC using a gradient of acetonitrile/water containing 0.1% trifluoroacetic acid. The identity of the purified material was checked by means of ion spray mass spectrometry.

### Example 6: Metal Complex Linker Assembly

### General Protocol for Ir/Ru Linker Conjugation:

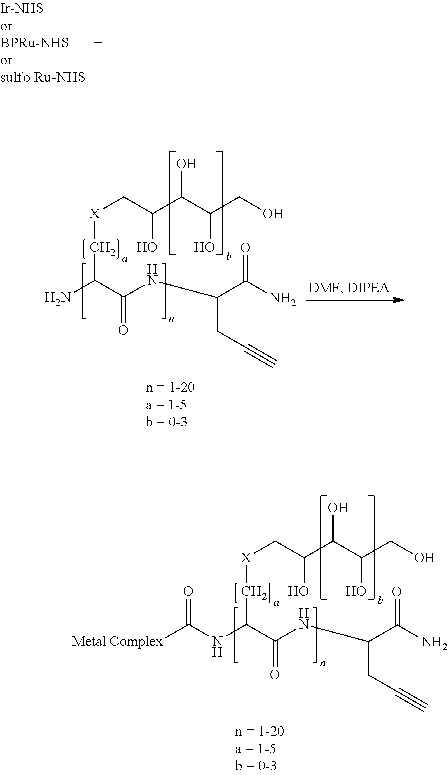

In 25 mL flask, the polyol linker (9.5 μmol, 1 eq.) was dissolved in 9 mL of dry DMF; the DIPEA (19 μmol, 2 eq.) and metal complex NHS ester (9.5 μmol, 1 eq.) (dissolved in 1 mL of DMF) were added. The reaction was left to react at r.t. overnight. After that, the solvent was evaporated in vacuo and the red solid obtained was dissolved in 2 mL of DMSO and the product was purified by HPLC-prep to give a red solid. X can be the same as in the definition mentioned above. X can be selected from the group consisting of O, S, CH2, SO, SO2 or 1,2,3-triazole, and amide.

HPLC Prep. Method for Ru Complexes:
Column C18 column, 10 mL/min flow:
0 min: 98% H₂O 0.05% TFA, 2% CH₃CN 0.05% TFA;
0-10 min: 98% H₂O 0.05% TFA, 2% CH₃CN 0.05% TFA;
10-60 min: 50% H₂O 0.05% TFA; 50% CH₃CN 0.05% TFA;
60-90 min: 0% H₂O 0.05% TFA; 100% CH₃CN 0.05% TFA;
   HPLC Prep. Method for Ir Complexes:
Column C18 column, 10 mL/min flow:
0 min: 98% H₂O; 2% CH₃CN;
0-10 min: 98% H₂O; 2% CH₃CN;
10-60 min: 50% H₂O; 50% CH₃CN;
60-90 min: 0% H₂O; 100% CH₃CN;

### Compound 20: BPRu-[FA41]₅-Pra-amid

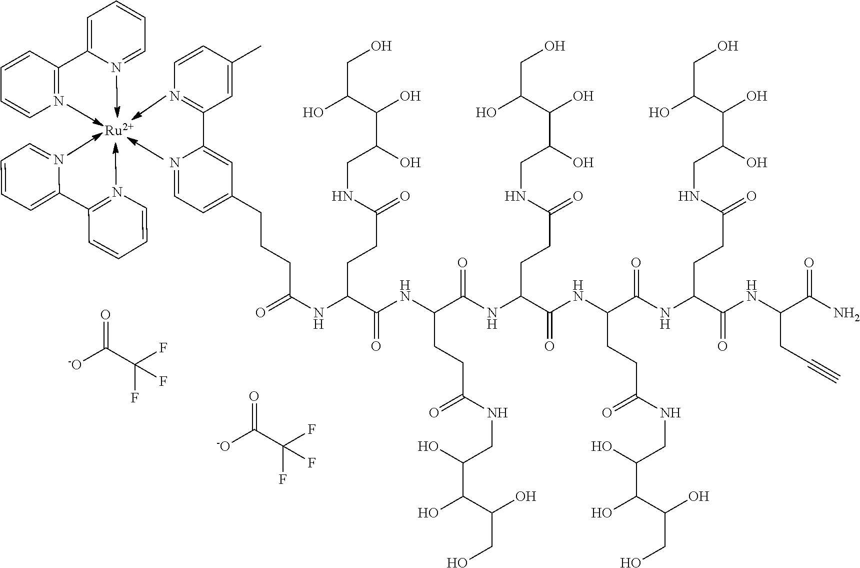

16.0 mg of product, 73% yield, red oil, TFA salt
HPLC-MS: (m/z) [M]²⁺/2 calcd 1037.40, found 1037.77.

### Compound 21: BPRu-[FA41]₁₀-Pra-amid

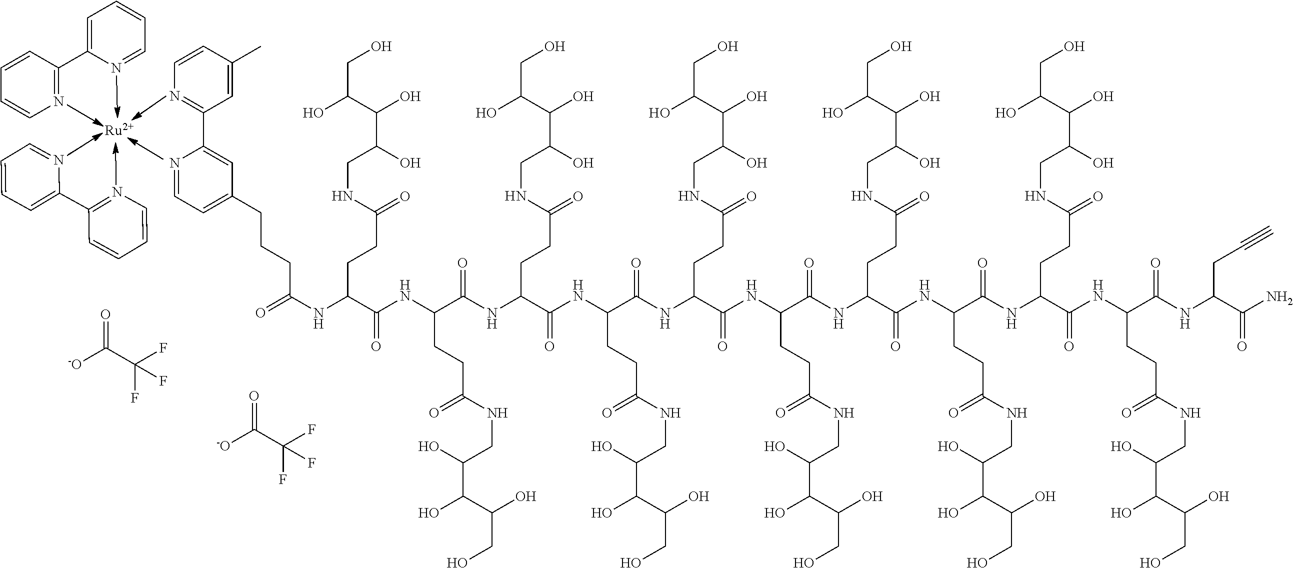

4.8 mg of product, 35% yield, red oil, TFA salt
HPLC-MS: (m/z) [M]²⁺/2 calcd 1692.69, found 1694.03; (m/z) [M+H]³⁺/3 calcd 1128.79, found 1129.05

### Compound 22: Ir-[FA41]₅-Pra-amid

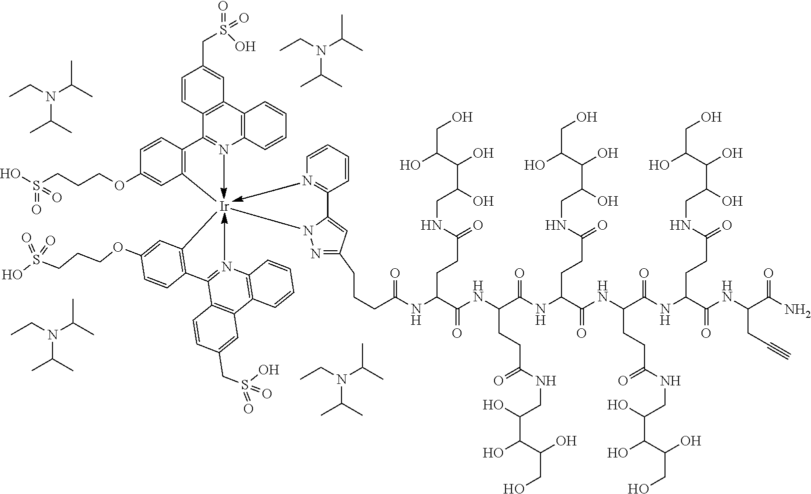

12.1 mg of product, 37% yield, red oil, DIPEA salt.
HPLC-MS: (m/z) [M+2H]²⁺/2 calcd 1400.91, found 1401.87

### Compound 23: Ir-[FA41]₁₀-Pra-amid

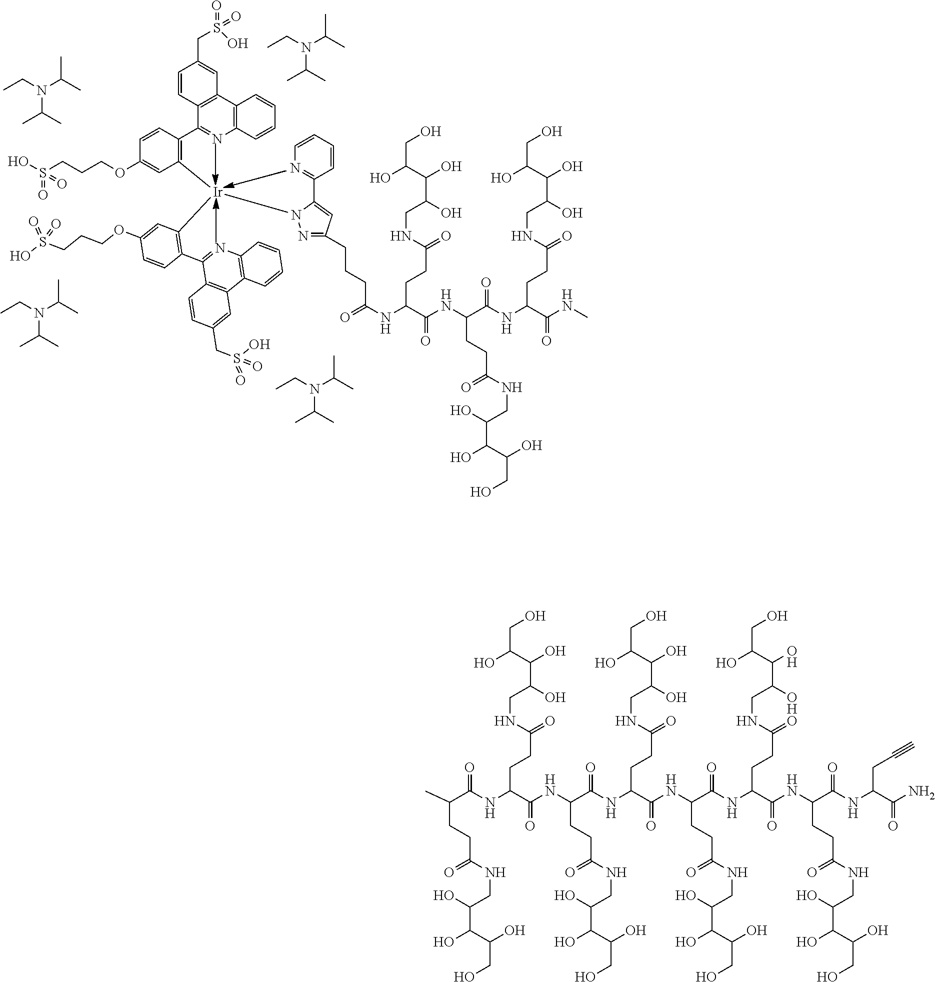

10.4 mg of product, 56% yield, red oil, DIPEA salt
HPLC-MS: (m/z) [M+3H]³⁺/3 calcd 1371.14, found 1371.74

### Compound 24: BPRu-[FA40]₅-Pra-amid

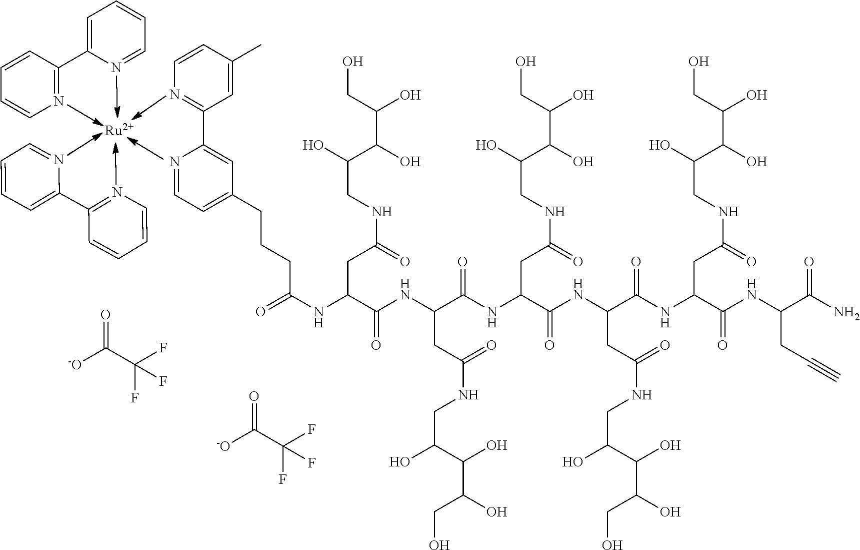

5.0 mg of product, 24% yield, red oil, TFA salt
HPLC-MS: (m/z) [M]²⁺/2 calcd 1002.36, found 1002.55.

### Compound 25: BPRu-[FA40]₁₀-Pra-amid

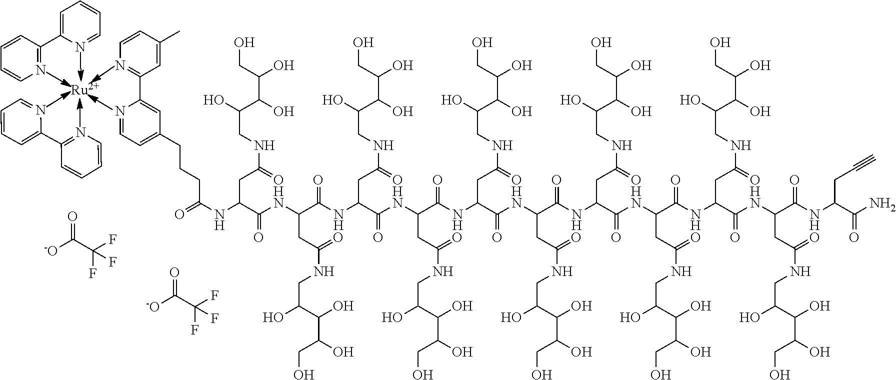

0.9 mg of product, 7% yield, red oil, TFA salt
HPLC-MS: (m/z) [M]²⁺/2 calcd 1622.61, found 1623.44; (m/z) [M+H]³⁺/3 calcd 1082.07, found 1082.75

### Compound 26: Ir-[FA40]₅-Pra-amid

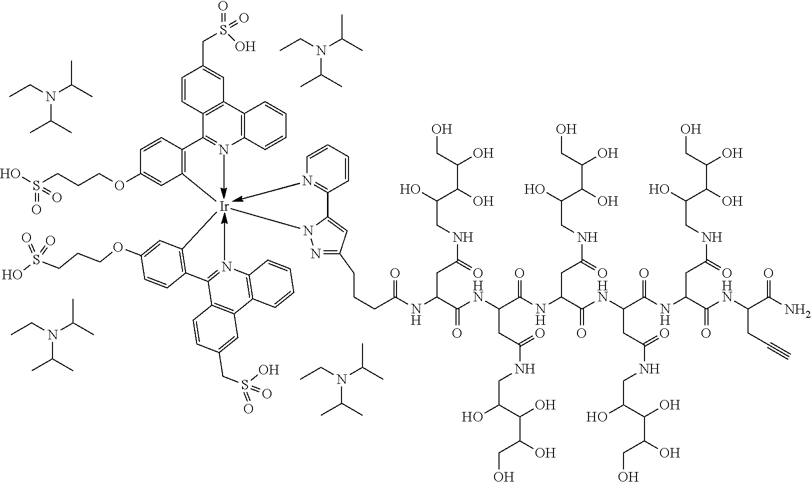

5.5 mg of product, 17% yield, red oil, DIPEA salt
HPLC-MS: (m/z) [M+2H]²⁺/2 calcd 1365.87, found 1366.82;

### Compound 27: Ir-[FA40]₁₀-Pra-amid

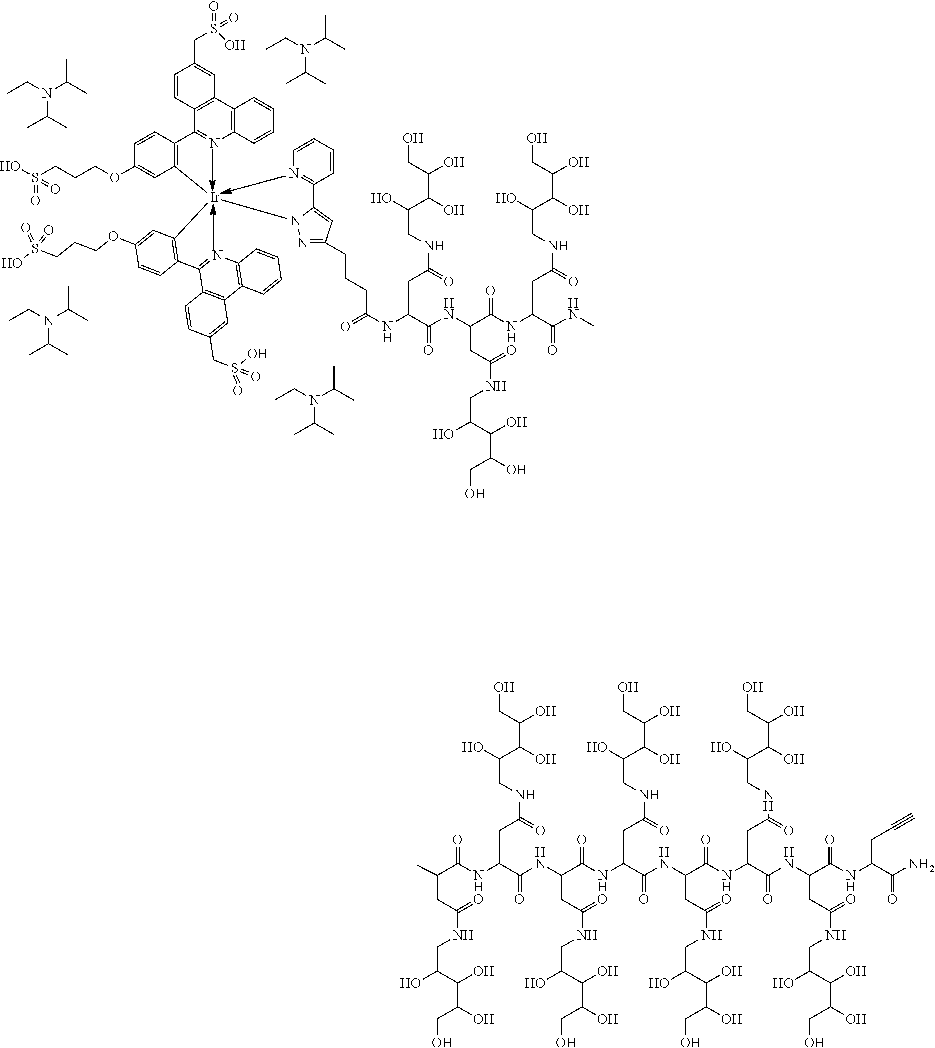

4.4 mg of product, 24% yield, red oil, DIPEA salt
HPLC-MS: (m/z) [M+3H]³⁺/3 calcd 1324.41, found 1125.08

### Compound 28: BPRu-[S773]5-K (MH)-NH2

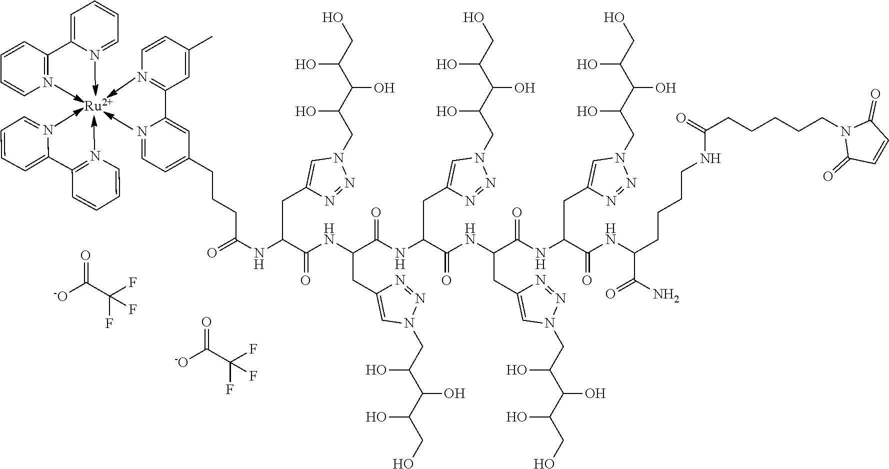

3.0 mg of product, 3% yield
HPLC-MS: (m/z) [M+2H]2+/2 calcd 1176.7, found 1176.7;
   Compound 29: sRu-[FA30]5-Pra-NH2

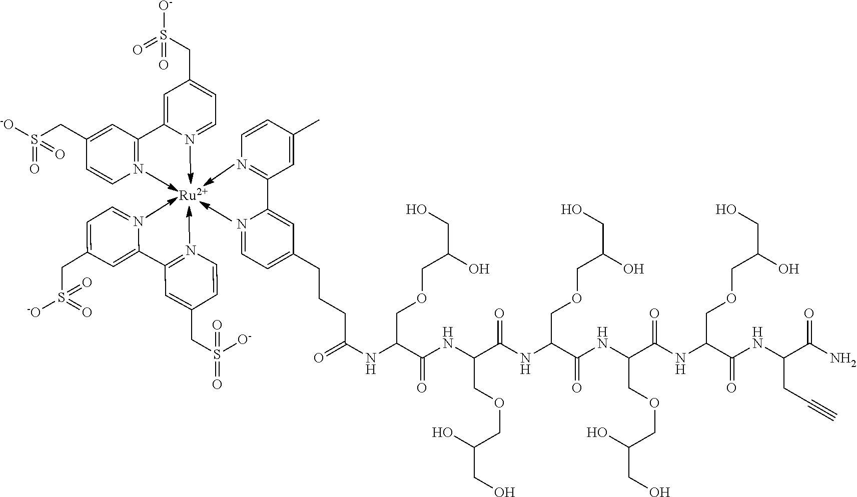

6.9 mg of product, 7% yield
HPLC-MS: (m/z) [M+2H]2+/2 calcd 974.0, found 973.8;

### Example 7: Non Site-Specific Conjugation of Labels

The Elecsys high sensitive Troponin-T (HS Tn-T) clone 5D8 was used for conjugation and ELC (electro-chemoluminescence) performance evaluation of the newly synthesized Ru (Ruthenium) or Ir (Iridium) complexes containing diverse polyol linkers. In case of labels with an alkyne functionality, the labeling was performed via click reaction. To generate clickable antibody, azide functionality was introduced to the IgG by conjugating Azide-PEG4-NHS at a stoichiometry of 1:5 or 1:10 (IgG: Label), giving MAB<Tn-T>chim-5D8-IgG-PEG4-N3 (1:5 or 1:10). This azido antibody was then conjugated with alkynyl-polyol-Ru/Ir labels via copper catalyzed click chemistry by using Tetrakis(acetonitrile) copper (1) tetrafluoroborate as copper 1 source and THPTA (Tris(3-hydroxypropyltriazolylmethyl)amine) as aqueous soluble copper ligand. The click reaction was performed in 50 mM KPP, 150 mM KCl, pH 7.4 with 5% Acetonitril.

In case of labels with a maleimido functionality, thiol functionalities were introduced to the IgG by conjugating with N-Succinimidyl-S-acetylthiopropionate (SATP) at a stoichiometry of 1:5 or 1:10 (lgG:SATP), giving MAB<Tn-T>chim-5D8-IgG-SATP (1:5 or 1:10). The acetyl protection from sulfur was removed by hydroxylamine treatment to get the final sulfhydryl-containing antibody. This SH-antibody was then conjugated with maleimido-polyol-Ru/Ir labels in 50 mM KPP, 150 mM KCl, pH 7.4 with 5% DMSO.

Conjugates of the Troponin-T (HS Tn-T) clone 5D8 with compound 30 and compound 31 were also obtained following the same procedure, and used as a comparison for the Elecsys performance

### Compound 30: BPRu-MEA

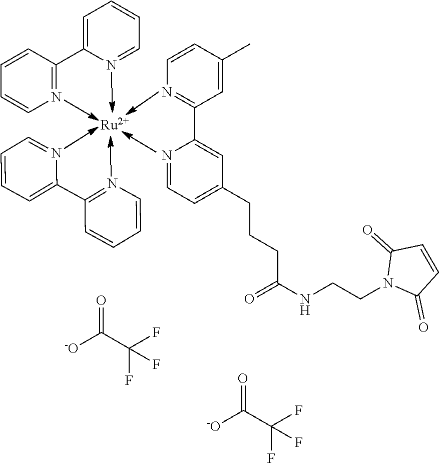

### Compound 31: BPRu-DADOO-PEG24-MH

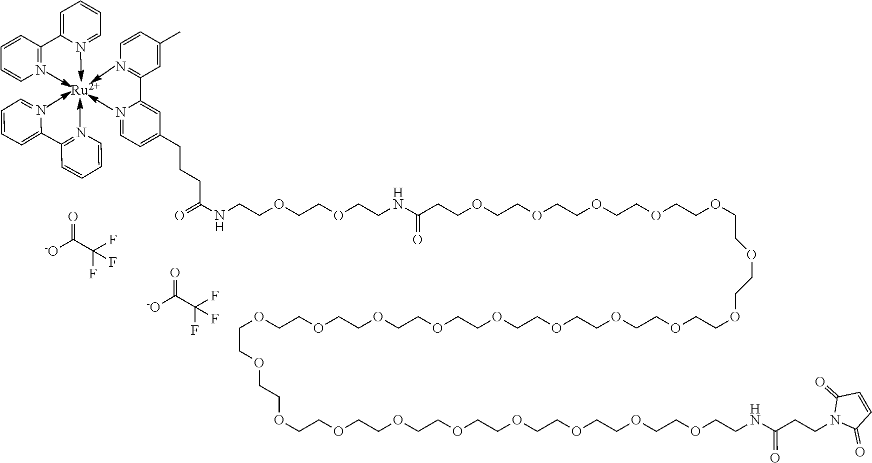

### Example 8: Elecsys Performance

All assays variants were run on a Cobas E170 Module using the Troponin Ths assay protocol with a blank control (Diluent Multiassay, Id. 11732277122, Roche Diagnostics GmbH, Mannheim, Germany, Cal1 and Cal2 from Troponon T hs CalSet (Id. 05092752190, Roche Diagnostics GmbH, Mannheim, Germany) using the Troponin T assay specifications.

These conjugates were then used in Troponin T hs Elecsys assay variants (Id. 05092744190, Roche Diagnostics GmbH, Mannheim, Germany) replacing the original R2 reagent at 2.5 μg/ml concentration. ECL measurement was carried out after one week incubation of the conjugated at 4° C. in TnT R2 buffer.

Incorporation rates of PEG- and polyol-based linkers are similar (see mean labelling rate). The new polyol linkers e.g. Ru-[FA41]10-Pra-NH₂ (compound 20) and Ir-[FA41]10-Pra-NH₂ (compound 23) show comparable signal-to-noise ratio in low (Cal1/MA) and high range (Cal2/MA) of TnT analyte when compared to the reference compounds 30 and 31. MA: serum blank; Cal1: 18 ng/L; Cal2: 4200 ng/L.

| | Mean | | | | | |
|---|---|---|---|---|---|---|
| | labelling | | | | | |
| (Label) stoichiometry | rate | MA | Cal1 | Cal2 | Cal1/MA | Cal2/MA |
| BPRu-MEA | 2.5 | 869 | 1167 | 262500 | 1.3 | 302 |
| (Compound 30) | | | | | | |
| BPRu-DADOO-PEG24- | 3.3 | 951 | 1152 | 255800 | 1.2 | 269 |
| MH | | | | | | |
| (Compound 31) | | | | | | |
| (BPRu-(S773)5- | 2.1 | 765 | 1323 | 175450 | 1.7 | 229 |
| K(MH)amid, MEA) 1:5 | | | | | | |
| (Compound 28) | | | | | | |
| (Ir-[FA41]10-Pra-NH₂) | 3.5 | 2248 | 3163 | 558479 | 1.4 | 248 |
| 1:7.5 | | | | | | |
| (Compound 23) | | | | | | |
| (BPRu-[FA41]10-Pra- | 3.2 | 727 | 1177 | 281553 | 1.6 | 388 |
| NH₂) 1:3.5 | | | | | | |
| (Compound 21) | | | | | | |

### Example 9: Conjugate Stability

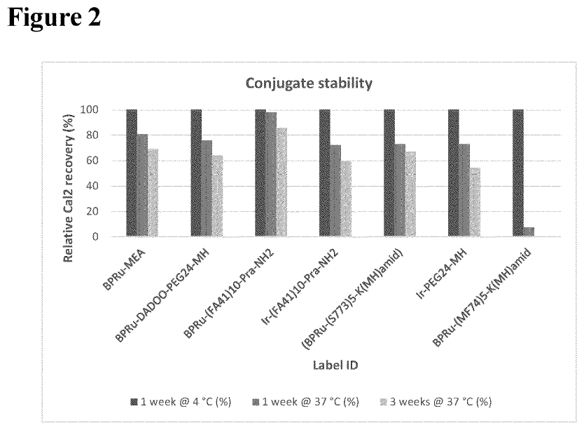

FIG. 2 shows the conjugate stability of different compounds as disclosed herein. Stability of conjugated was evaluated by measuring the ECL signal following incubation of the samples for 1 week and 3 weeks at 35° C. after the first measurement. The signal recovery was calculated by comparing the obtained signal with the one of sample incubated at 4° C. for one week. New labels (Ir-[FA41]10-Pra-NH₂) (compound 23) and (Ru-[FA41]10-Pra-NH₂) (Compound 21) showed significantly improved stability over last generation polyol-based labels (BPRu-(MF74) 5-K (MH) amid). In addition to that, comparison between linkers conjugated through maleimide chemistry and linkers conjugated by click chemistry shows an improved stability of the latter over time.

| Structure: | |
|---|---|
| BPRu- (MF74)5- K(MH) amid | 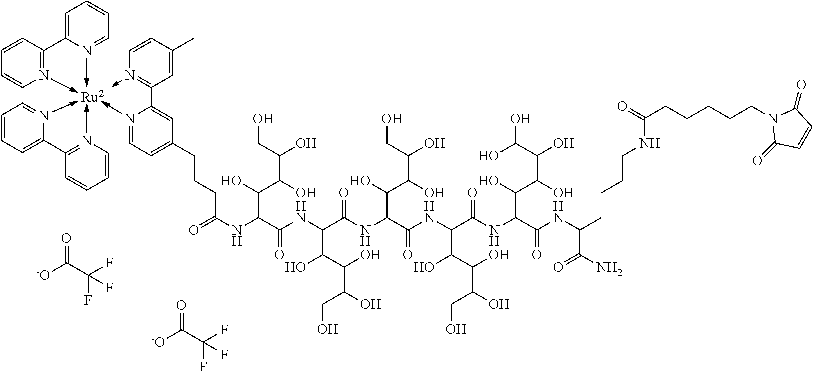 |
| Ir- PEG24- MH | 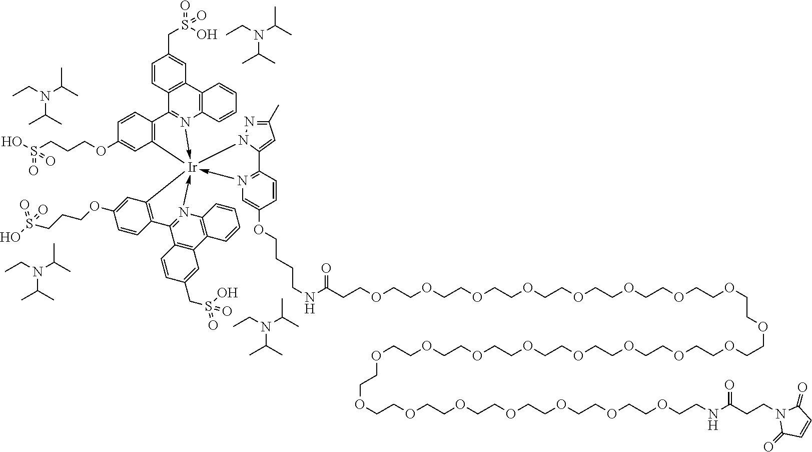 |

This patent application claims the priority of the European patent application 22157541.8, wherein the content of this European patent application is hereby incorporated by references.

1. A method for detecting an analyte of interest in a sample comprising the steps of
a) providing the sample comprising the analyte of interest;
b) providing a complex comprising a linker, wherein the linker covalently binds a label compound and an analyte-specific binding agent, wherein the label compound is capable of producing a detectable signal; more preferably an electrochemiluminescence based signal,
c) coupling the sample of step a) with the complex of step b); and
d) detecting the analyte of interest by using the detectable signal of the label compound,
wherein the complex is a compound of formula I: 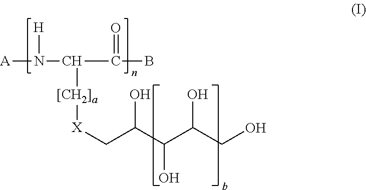
wherein A represents the label compound and B represents the analyte-specific binding agent or vice versa,
X is selected from the group consisting of O, S, CH₂, SO, SO₂, 1,2,3-triazole, and amide,
a is 1 to 5,
b is 0 to 3, and
n is an integer from 2 to 20.

2. The method of claim 1, wherein the complex is selected from of at least one compound having at least one of the following formulae 4-I to 4-X:

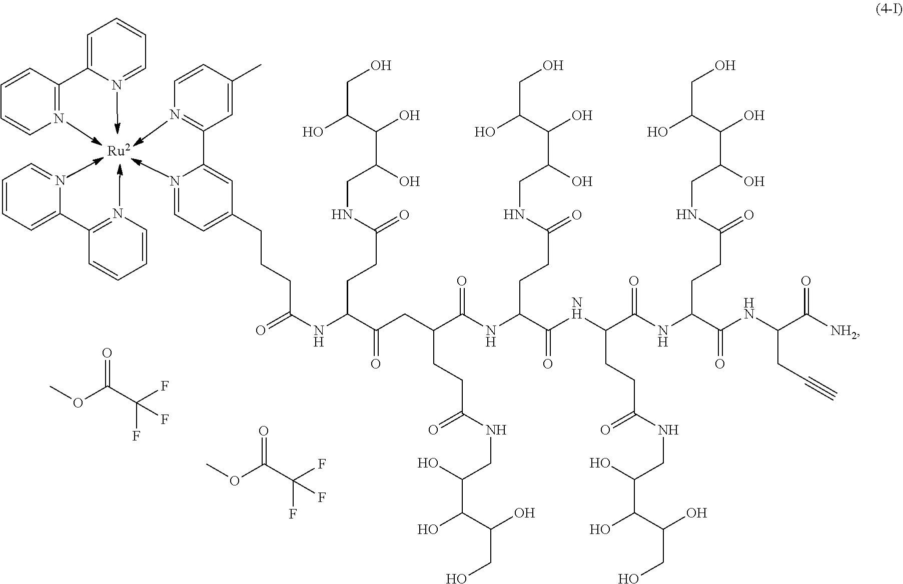

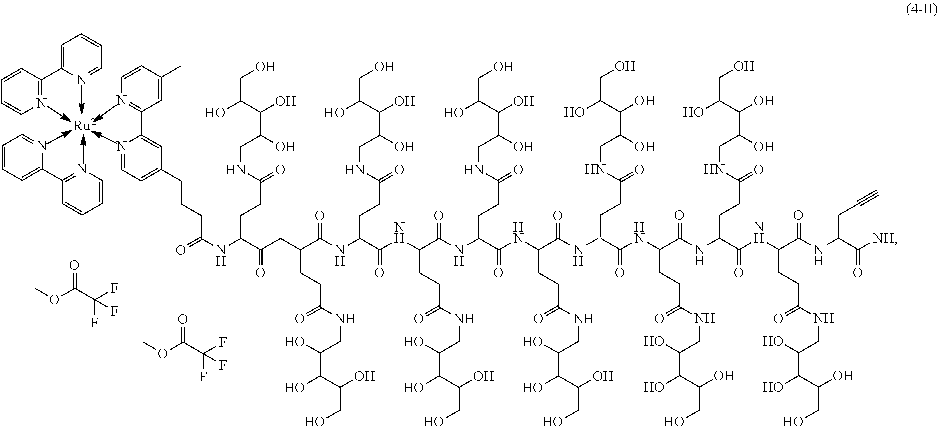

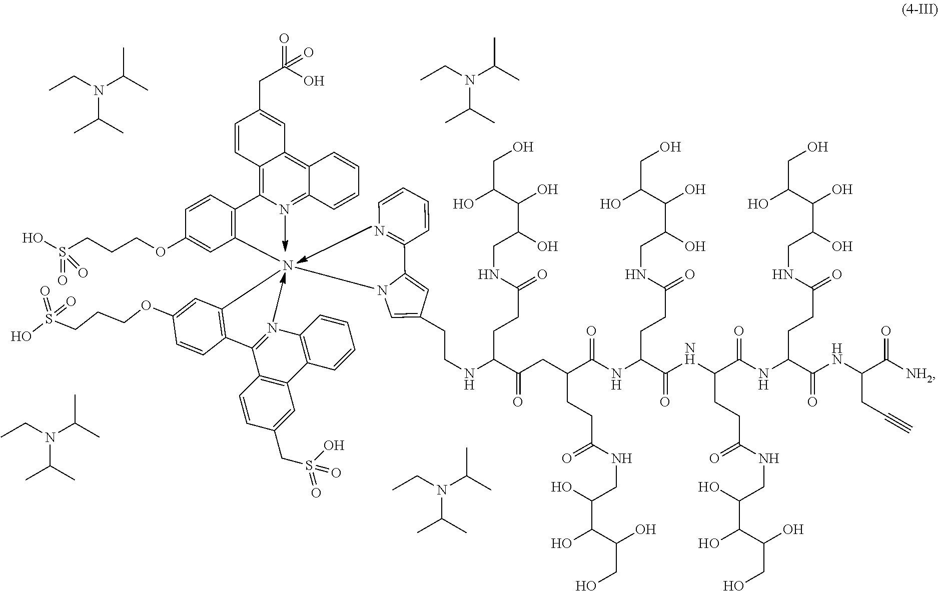

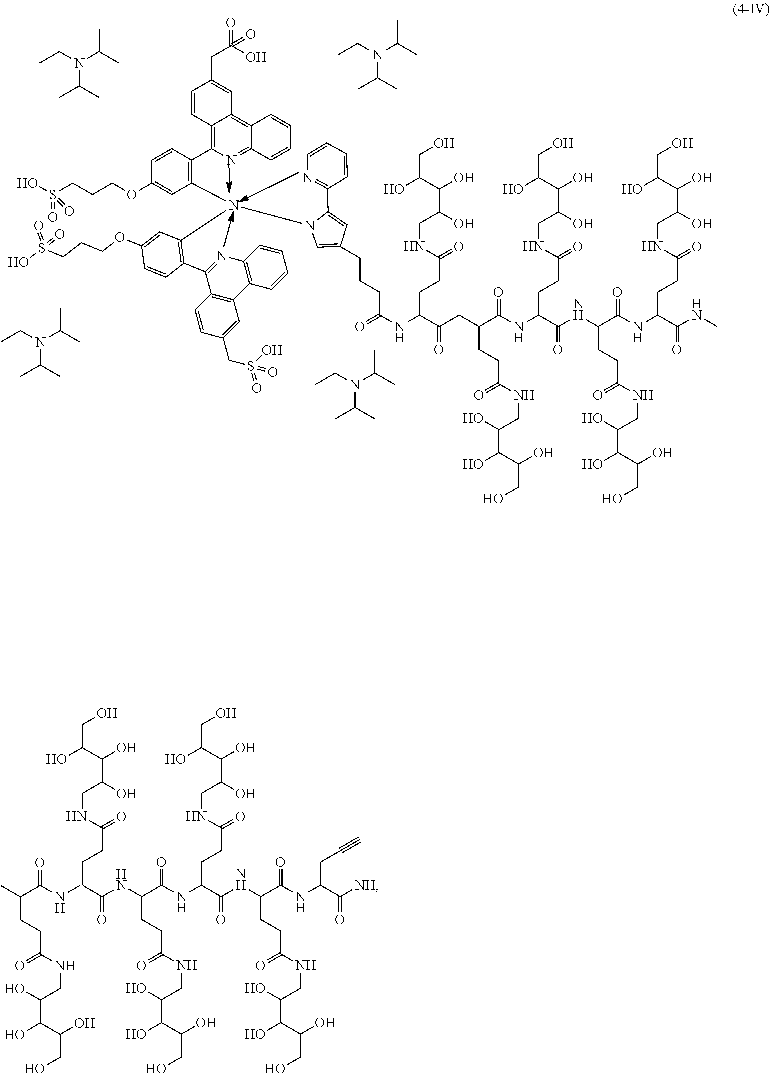

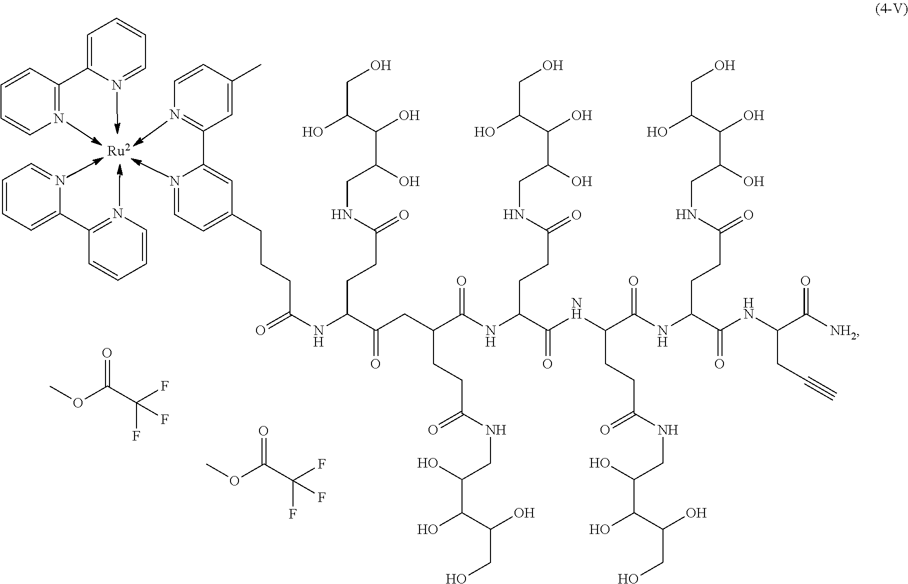

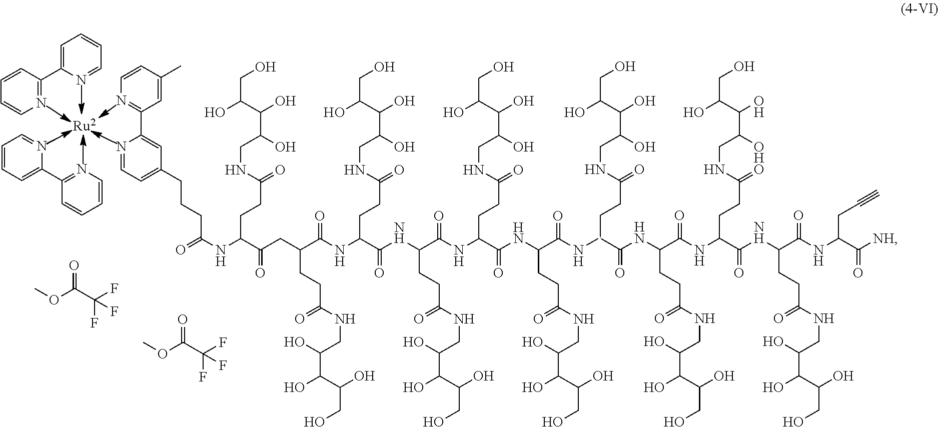

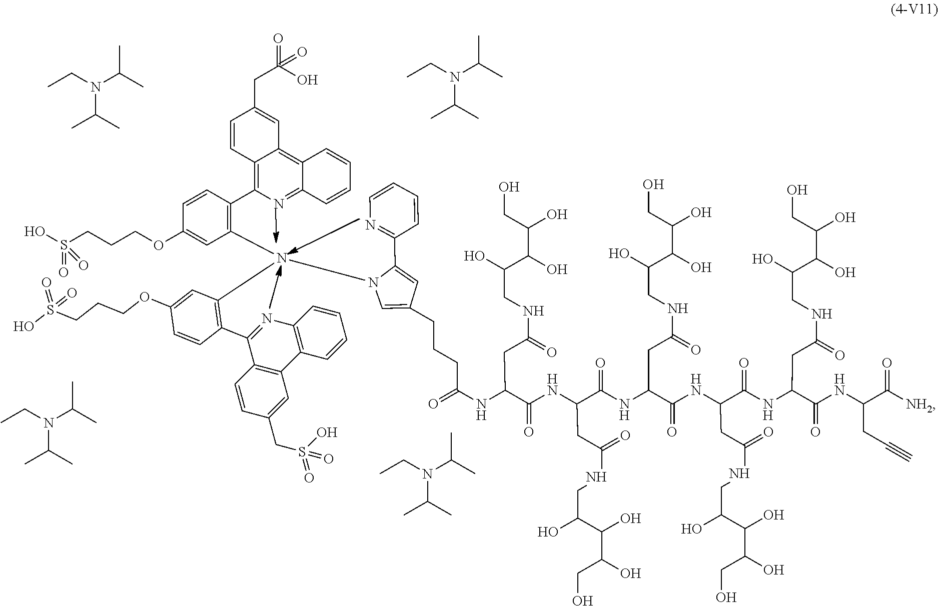

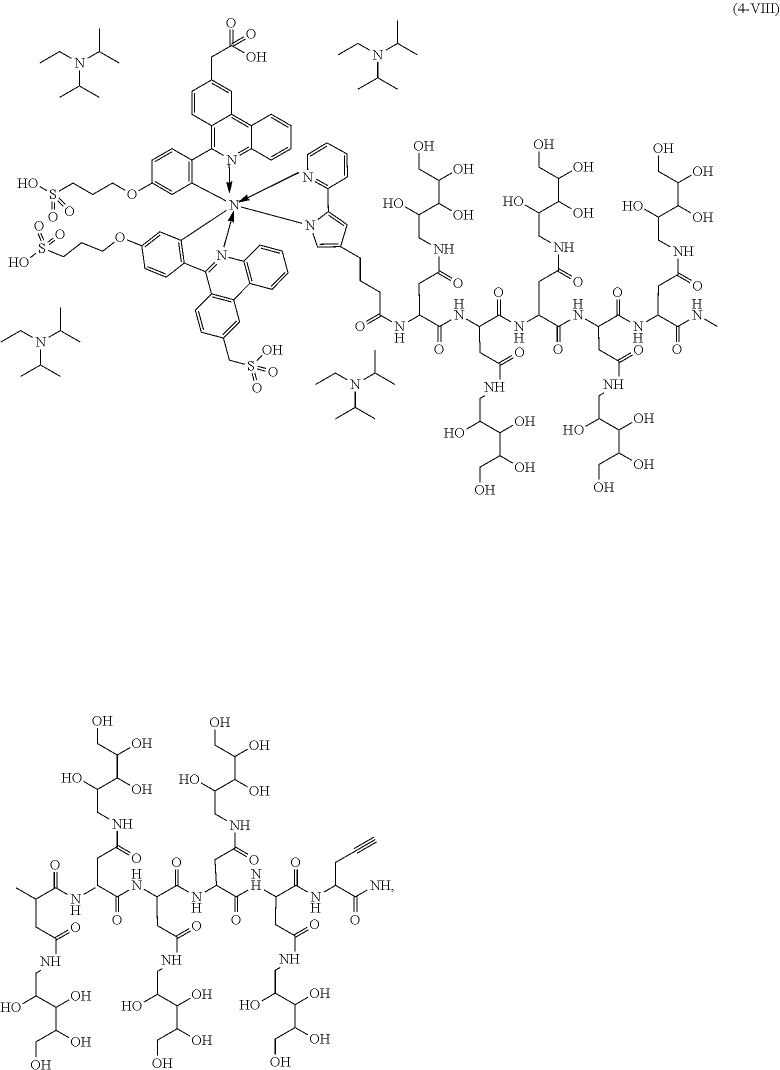

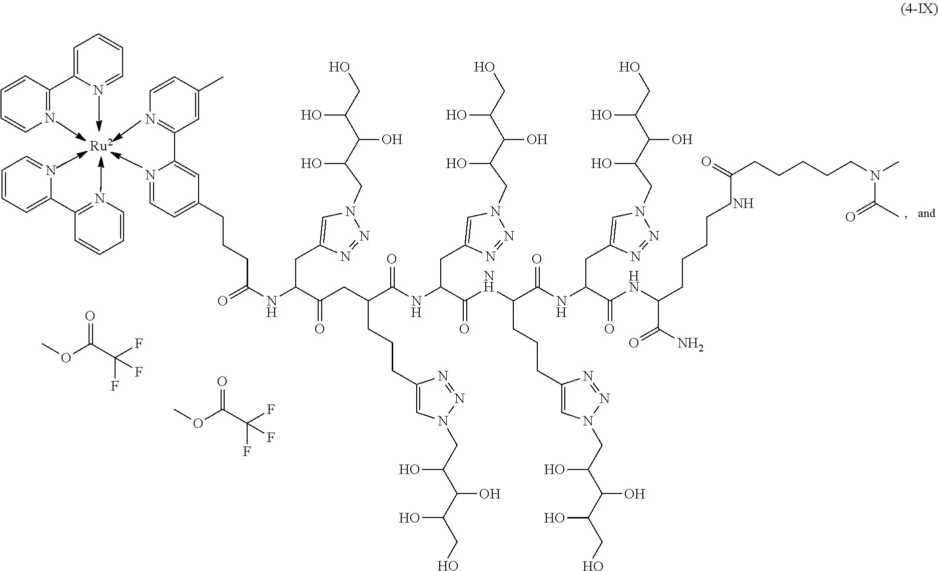

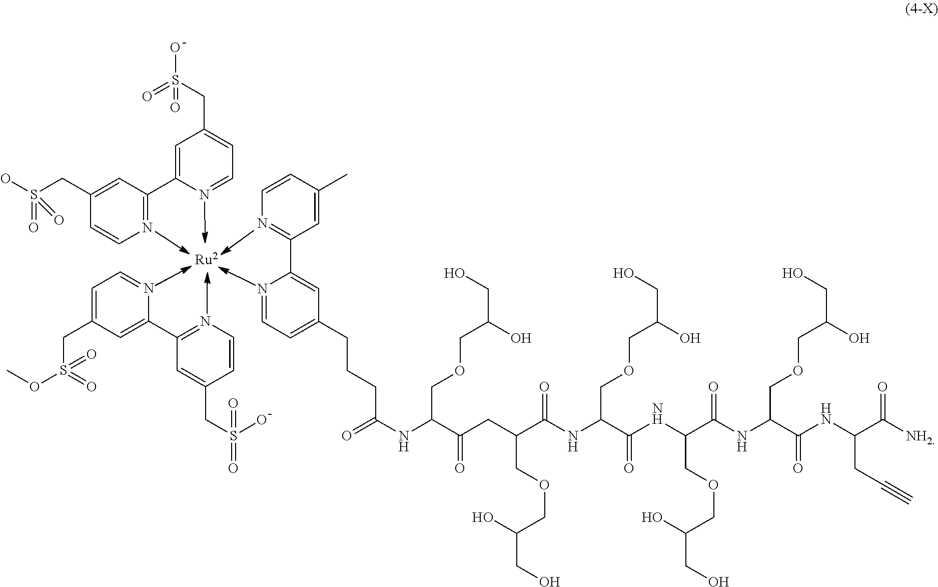

3. The method of claim 1, wherein the label compound is selected from the group consisting of an enzyme, a fluorescent dye, a luminescent dye, a metal chelate complex, and a moiety containing a radioisotope.

4. The method of claim 1, wherein the label compound comprises a metal ion, which is Ru²⁺ or Ir³⁺.

5. The method of claim 1, wherein the analyte-specific binding agent is selected from the group consisting of an antibody, an analyte-specific fragment and/or derivative of an antibody, an aptamer, a spiegelmer, a darpin, a lectin, an ankyrin repeat containing protein, and a Kunitz type domain containing protein.

6. The method of claim 1, wherein prior, during or after step (c) the analyte is immobilized on a solid phase.

7. The method of claim 1, wherein step b) comprises a peptide-based synthesis, preferably a solid phase peptide synthesis (SPPS).

8. (canceled)

9. A kit for performing detection of an analyte of interest in a sample, the kit comprising in separate containers
a) a solid phase capable of immobilizing the analyte; and
b) a compound of formula I: 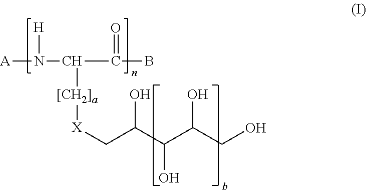
wherein A represents the label compound and B represents the analyte-specific binding agent or vice versa,
is selected from the group consisting of O, S, CH₂, SO, SO₂ or 1,2,3-triazole, and amide,
a is 1 to 5,
b is 0 to 3, and
n is an integer from 1 to 20 or from 2 to 20.

10. (canceled)

11. A complex of formula I, 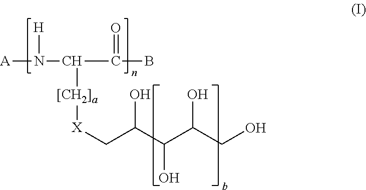
wherein A represents a label compound and B represents an analyte-specific binding agent or vice versa,
X is selected from the group consisting of O, S, CH₂, SO, SO₂ or 1,2,3-triazole, and amide,
a is 1 to 5,
b is 0 to 3, and
n is an integer from 1 to 20 or from 2 to 20.

12. A method to synthesize a complex of claim 11, the method comprising the steps of
a) providing a monomer or derivatives thereof, wherein the monomer is an amino acid comprising an amino group, a carboxy group and at least two hydroxyl group, wherein the amino group or the carboxy group is protected by a first protecting group, and the at least one or each hydroxyl group is protected by a second protecting group; and
b) using the monomer in a process of solid phase peptide synthesis, cleaving the first and second protection group and forming a complex of formula I 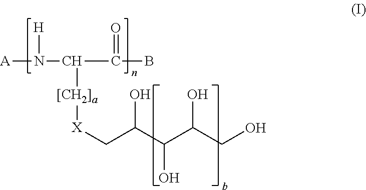
wherein A represents the label compound and B represents the analyte-specific binding agent or vice versa,
X is selected from the group consisting of O, S, CH₂, SO, SO₂ or 1,2,3-triazole, and amide,
a is 1 to 5,
b is 0 to 3, and
n is an integer from 1 to 20 or from 2 to 20.

13. A monomer used for a peptide-based synthesis comprising the following formula II 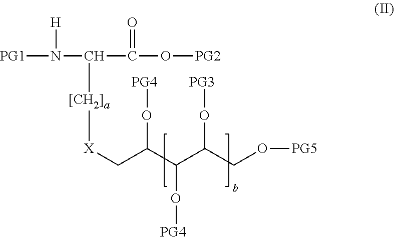
wherein PG1, PG2, PG3, PG4, PG5, PG6 are each independently a protecting group,
wherein X is selected from the group consisting of O, S, CH₂, SO, SO₂ or 1,2,3-triazole, and amide,
wherein a is 1 to 5,
wherein b is 0 to 3.

14. The monomer of claim 13, wherein at least two of the protecting groups selecting from the group consisting of PG1, PG2, PG3, PG4, PG5, PG6 are linked to each other, for example by at least one covalent bond.

15. The method of claim 1, wherein the detectable signal is a chemiluminescence based signal or an electrochemiluminescence based signal.

16. The complex of claim 11, wherein the compound is suitable to detect an analyte of interest in a sample.

17. The monomer of claim 13, wherein if PG1 is a protecting group, PG2=H or vice versa.

18. The method of claim 1, wherein a is 1 to 4 or 1 to 2.

19. The kit of claim 9, wherein a is 1 to 4 or 1 to 2, and/or n is an integer from 1 to 15 or from 2 to 15.

20. The complex of claim 11, wherein a is 1 to 4 or 1 to 2, and/or n is an integer from 1 to 15 or from 2 to 15.

21. The method of claim 12, wherein in step b) a is 1 to 4 or 1 to 2, and/or n is an integer from 1 to 15 or from 2 to 15.

22. The monomer of claim 13, wherein a is 1 to 4 or 1 to 2.
